(19) United States
(12) Patent Application Publication
(10) Pub. No.: US 2004/0082521 A1
(43) Pub. Date: Apr. 29, 2004

(54) Novel formulations of digitalis glycosides for treating cell-proliferative and other diseases

(75) Inventor: Chandra U. Singh, San Antonio, TX (US)

Correspondence Address: Daniel S. Hodgins, Suite 2100, 112 East Pecan Street, San Antonio, TX, 78205, US

(73) Assignee: Azaya Therapeutics Inc.

(21) Appl. No.: 10/404,902

(22) Filed: Mar. 31, 2003

Related U.S. Application Data
(60) Provisional application No. 60/368,880, filed on Mar. 29, 2002.

(51) Int. Cl. A61K 31/704; A61K 9/127

(52) U.S. Cl. 514/26; 424/450

(57) ABSTRACT

The present invention provides methods, preparations, and uses of a variety of liposomal-digitalis glycoside compositions. The present invention also provides protein-stabilized nanoparticle formulations containing liposomal-digitalis glycosides such as Oleandrin, digitoxin, and digoxin with reduced toxicity, high drug to lipid ratio, long circulating time in the bloodstream and the ability to deliver the drug to tumor sites. In another aspect, the present invention provides an effective method to reduce the growth of cancers or reduce the incidence of metastases.

### FIELD OF THE INVENTION

The present invention is generally directed to the fields of medicine and pharmacology and is specifically related to a pharmaceutical compositions, containing Oleandrin derived from the plant Nerium Oleander L. and other cardiac glycosides, for use in the treatment of the cell-proliferative diseases including cancer, AIDS and other diseases such as diabetes and cardiac disorders.

In another aspect, the present invention provides method, preparation and use of a variety of protein microspheres, liposomal and protein stabilized liposomal formulations of Oleandrin and cardiac glycosides with reduced toxicity, high drug to lipid ratio, long-circulating time in the bloodstream and able to deliver the drug to the desired sites such as tumor sites. The present invention also provides an effective method to reduce the growth of cancers or reducing the incidence of metastases.

### BACKGROUND OF THE INVENTION

Nerium Oleander is an evergreen shrub reaching four meters in height. Leaves are 10 to 22 cm long, narrow, untoothed and short-stalked, dark or grey-green in color. Some cultivars have leaves variegated with white or yellow. All leaves have a prominent mid rib, are “leathery” in texture and usually arise in groups of three from the stem. The plant produces terminal flower heads, usually pink or white, however, 400 cultivars have been bred and these display a wide variety of different flower color: deep to pale pink, lilac, carmine, purple, salmon, apricot, copper, orange and white (Huxley 1992). Each flower is about 5 cm in diameter and five-petalled. The throat of each flower is fringed with long petal-like projections. Occasionally double flowers are encountered amongst cultivars. The fruit consists of a long narrow capsule 10 to 12 cm long and 6 to 8 mm in diameter; they open to disperse fluffy seeds. Fruiting is uncommon in cultivated plants.

The plant exudes a thick white sap when a twig or branch is broken or cut (Font-Quer 1974; Schvartsman 1979; Lampe & McCann 1985; Peam 1987). Where the species grows in the wild (i.e. in the Mediterranean), it occurs along watercourses, gravely places and damp ravines. It is widely cultivated particularly in warm temperate and subtropical regions where it grows outdoors in parks, gardens and along road sides. Elsewhere, where the plant is not frost-tolerant (e.g. in central and western Europe), it may be grown as a conservatory or patio plant. N. Oleander is cultivated worldwide as an ornamental plant; it is native only in the Mediterranean region (Kingsbury 1964; Hardin & Arena 1974).

In Mediterranean region, the plant has been used extensively for medicinal purposes. For example, the macerated leaves have been used for itch and fall of hair. The fresh leaves have been applied on tumors for treatment. The decoction of leaves and bark has been used as antisyphillic. The decoction of leaves has been used as a gargle to strengthen the teeth and gum and as a nose drop for children (Dymock 1890; Chopra 1956; Dey 1984; Kirtikar 1987).

Oleander is one of the digitalis-like plants. These plants produce certain steroidal glycosides with cardiac properties, called as either digitalis glycosides or cardiac glycosides. Digitalis glycosides are one of the most useful groups of drugs in therapeutics (Melero 2000). Among the different digitalis glycosides present in Digitalis purpurea, digoxin and its derivatives (acetyl- and methyl-digoxin) are the digitalis glycosides most currently used in therapeutics.

The oleander plant has certain toxic properties due to the presence of digitalis glycosides such as Oleandrin. It is estimated that as many as 100 novel chemical substances are present in various parts of the Oleander plant (Krasso 1963; Siddiqui 1987-1995; Taylor 1956; Abe 1992; Hanada 1992). Oleandrin [C₃₂H₄₈O₉], a glycoside, is the main toxin in the plant. Its chemical name is 16b-acetoxy-3b-[(2,6-dideoxy-3-0-methyl-a2-L-arabino-hexopyranosyl) oxy]14-hydroxy-5β, 14β-card-20(22)-enolide (Reynolds 1989). Oleandrin forms colorless, odorless, acicular crystals which are very bitter (Shaw & Pearn 1979). The concentration of Oleandrin in the plant tissues is approximately 0.08% (Schvartsman 1979). Oleandrin is almost insoluble in water; it has little resistance to light but it is heat-stable (Pearn 1987; Reynolds 1989). The chemical structure of Oleandrin is provided in Formula I.

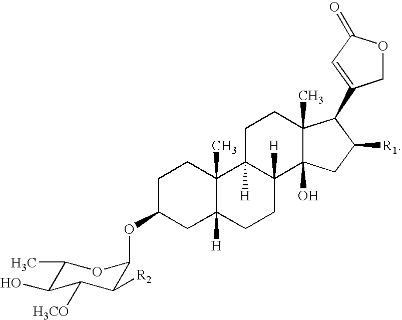

Formula I.

| | |
|---|---|
| 1. Oleandrin: R1 = OCOCH3; | R2 = H |
| 2. Neriifolin: R1 = H; | R2 = OH |
| 3. Odoroside A: R1 = H; | R2 = H |
| 4. Odoroside H: R1 = H; | R2 = OH |

When ingested, Oleandrin gets widely distributed in the body and high concentrations of Oleandrin have been measured in blood, liver, heart, lung, brain, spleen and kidney in a fatal case of N. Oleander extract poisoning (Blum & Rieders 1987). Oleandrin is eliminated very slowly from the body (one to two weeks) (Shaw & Pearn 1979). In 1957, the National Cancer Institute showed that three compounds in the plant, namely, Oleandrin, adynerin and ursolic acid had significant anti-cancer activities on various cancer cell lines.

Since then several new chemical compounds have been identified from the methanolic or ethanolic extracts of the plant. Oleandrin is also present other plants like Operculina turpethum which is called by the common name Nishotra in India.

The U.S. Pat. No. 5,135,745 describes a procedure for the preparation of the extract of the plant in water. The extraction of the plant Nerium Oleander involves, cooking the leaves and stems of the plant in water for 2-3 hours and filtering off the residues. The chemical constituents of the aqueous extract have been analyzed. It has been found to contain several polysaccharides with molecular weights varying from 2 KD to 30 KD, Oleandrin, Oleandrogenin and proteins (Wang 2000). It has been shown that the water extract of the plant and Oleandrin were able to induce cell killing in human cancer cells, but not in murine cancer cells and the cell-killing potency of Oleandrin was greater than that of the water extract. Canine oral cancer cells treated with water extract showed intermediate levels of response, with some abnormal metaphases and cell death resulting from the treatment (Pathak 2000)

Cardiac glycosides are used clinically to increase contractile force in patients with cardiac disorders. A list of cardiac glycosides from plants and toads are given in Table 1.

TABLE 1
Fanerogam and Toad species containing digitalis glycosides.

| Species | Cardiotonic glycosides |
|---|---|
| 1. Family Apocynaceae | |
| Nerium oleander | Oleandrin, neriin, neriantin. |
| Nerium odorum | Odoroside A and B. |
| Strophantus gratus, S. kombe, | Ouabain (G-strophantin), |
| S. his-pidus, | cymarin, sarmentocymarin, |
| S. sarmentosus, S. emini | periplocymarin, K-strophantin. |
| Acokanthera schimperi (A. | Ouabain. |
| ouaba\ddot{i}o), A. venenata, A. | |
| abyssinica | |
| Thevetia nereifolia | Thevetin, cerberin, peruvoside. |
| Thevetia yecotli | Thevetosin, thevetin A. |
| Cerbera odollam | Cerberin. |
| Cerbera tanghin | Tanghinin, |
| | deacetyltanghinin, cerberin. |
| Adenium boehmanianum | Echujin, hongheloside G. |
| 2. Family Asclepiadaceae | |
| Periploca graeca | Periplocin. |
| Periploca nigrescens | Strophantidin, strophantidol, |
| | nigrescin. |
| Xysmalobium undulatum | Uzarin. |
| Gomphocarpus fruticosus | Uzarin. |
| Calotropis procera | Calotropin. |
| 3. Family Brassicaceae | |
| Cheiranthus cheiri | Cheiroside A, cheirotoxin. |
| 4. Family Celastraceae | |
| Euonymus europaeus, E. atropur- | Eounoside, euobioside, euomonoside. |
| Pureus | |
| 5. Family Crassulaceae | |
| Kalanchoe lanceolata | Lancetoxin A and B. |
| Kalanchoe tomentosa | Kalanchoside. |
| Kalanchoe tubiflorum | Bryotoxin A-C. |
| Kalanchoe pinnatum | Bryotoxin C, bryophyllin B. |
| Tylecodon wallichii | Cotiledoside. |
| Tylecodon grandiflorus | Tyledoside A-D, F and G. |
| Cotyledon orbiculata | Orbicuside A-C. |
| 6. Family Fabaceae | |
| Coronilla sp. | Alloglaucotoxin, corotoxin, |
| | coroglaucin, glaucorin. |
| 7. Family Iridaceae | |
| Homeria glauca | Scillirosidin derivatives. |
| Moraea polystachya, | Bovogenin A derivatives. |
| M. graminicola | |
| 8. Family Liliaceae | |
| Urginea scilla, U. maritima | Scillarene A and B, scilliroside, |
| | scillarenia, scilliacinoside, |
| | scilliglaucoside, scilliglaucosidin, |
| | scil-liphaeosidin, scilliphaeoside, |
| | scillirosidin, scillirubrosidin, |
| | scillirubroside, proscillaridin A. |
| Urginea rubella | Rubelin. |
| Convalaria majalis | Convalloside, convallatoxin. |
| Bowiea volubilis, B. kilimand- | Bovoside A, glucobovoside A, |
| Scharica | bovoruboside. |
| 9. Family Moraceae | |
| Antiaria africana, A. toxicaria | Antiarin a. |
| 10. Family Ranunculaceae | |
| Helleborus niger, H. viridis, | Helleborein, helleborin, hellebrin. |
| H. foeti Dus | |
| Adonis vernalis, A. aestivalis, A. | Adonidin, adonin, cymarin, |
| autumnalis, A. flammea | adonitoxin. |
| 11. Family Santalaceae | |
| Thesium lineatum | Thesiuside. |
| 12. Family Scrophulariaceae | |
| Digitalis purpurea, D. lanata | Digitoxin, gitoxin, gitalin, digoxin, |
| | F-gitonin, digitonin, lanatoside A-C. |
| 13. Toad Species Genins | |
| Bufo vulgaris | Bufotalin, bufotalinin, bufotalidin. |
| Bufo japonicus | Gamabufagin. |
| Bufo gargarizans | Cinobufagin. |
| Bufo marinus | Marinobufagin. |
| Bufo arenarum | Arenobufagin. |
| Bufo regularis | Regularobufagin. |
| Bufo valliceps | Vallicepobufagin. |
| Bufo quercicus | Quercicobufagin. |
| Bufo viridis | Viridibufagin. |
| Bufo sp. | Pseudobufotalin. |

Their mechanism of action is well established and involves inhibition of the plasma membrane Na⁺, K⁺-ATPase, leading to alterations in intracellular K⁺ and Ca²⁺ levels.

Na⁺, K⁺-ATPase (EC 3.6.1.37) or sodium pump, is a carrier enzyme present in almost every animal cell and was discovered by Skou in 1957. Its physiological function is to maintain the Na⁺ and K⁺ electrochemical gradients through the cell membrane, keeping low Na⁺ and high K⁺ intracellular concentrations. Such concentrations of ions, their gradients and the consequent membrane potential determine a broad range of cellular functions, as excitability of nerves and muscle cells, secondary active transport and cellular volume regulation. It is estimated to consume about 25% of total ATP consumed at rest.

Related to the transport activity, the enzyme takes out 3 Na⁺ in exchange for 2 K⁺ carried into the cell. So, it allows the restoration of the appropriate Na⁺:K⁺ ratio to maintain the transmembrane difference of potential (Na⁺ and K⁺ concentrations at rest are: [Na⁺]int.=7-20 mM, [Na⁺]ext.=140 mM, [K⁺]int.=110- 120 mM, [K⁺]ext.=4-5 mM). It requires ATP and Mg²⁺0 for activity. Binding of ligands to the enzyme, including a phosphorylation step, leads to conformational changes associated to Na⁺ and K⁺ transport. The supposed mechanism of action currently accepted was firstly proposed by Albers (1967) and Post (1969). This mechanism includes a step in which the enzyme, after leaving out 3 Na⁺ and before taking in 2 K⁺, can be bound, and thus inhibited, by digitalis glycosides or their analogues, preventing K⁺ binding and then stopping enzyme activity.

Na⁺, K⁺-ATPase is regulated by Na⁺ and K⁺ concentrations, as well as by several hormones, as aldosterone, thyroid hormones, catecholamines and peptide hormones (vasopresin or insulin). Hormone regulation can be carried out at different levels, from cell surface to nucleus, and it can be expressed at short or long term (Geering 1997).

Digitalis glycosides can be defined as allosteric inhibitors of Na⁺, K⁺ ATPase, and are not covalently bound to the enzyme (Repke 1989). According to the still most widely accepted mechanism of action for digitalis glycosides (Thomas 1990), they act through inhibition of Na⁺, K⁺-ATPase, thus raising indirectly the intracellular Ca²⁺ concentration ([Ca²⁺]i). Therapeutic concentrations of digitalis glycosides produce a moderate enzyme inhibition (about 30%). When the cell is depolarised, there is a lower amount of enzymes available for the restoration of the Na⁺/K⁺ balance. The remaining enzymes, non-inhibited, will act faster, because the high [Na⁺]i and the ionic balance must be restored before the following depolarisation, although it will take longer than if every enzyme were available. This lag causes a temporary increase of [Na⁺]i, reaching higher concentrations than if ATPase activity were not partially inhibited. This temporary increase of [Na⁺]i, modifies [Ca²⁺]i through a Na⁺/Ca²⁺ exchanger which allows Na⁺ exit from the cell in exchange for Ca²⁺, or Ca²⁺ exit from the cell in exchange for Na⁺, depending on the prevailing Na⁺ and Ca²⁺ electrochemical gradients (Blaustein 1974). This mechanism decreases exchange rate, or even reverses exchanger ion transport, being Ca²⁺ carried into the cell; anyway increasing [Ca²⁺]i and thus increasing contractile force.

When the concentration of digitalis glycosides reaches toxic levels, enzyme inhibition is too high (>60%), thus decreasing Na⁺ and K⁺ transport to the extent that the restoring of normal levels during diastole is not possible before the next depolarisation. Then, a sustained increase of [Na⁺]i , and thus of [Ca²⁺]i, gives rise to toxic effects (i.e. arrhythmia) of these glycosides.

Digitalis glycosides represent a very important group of drugs for the treatment of heart failure, but display a main disadvantage, which arises from their narrow therapeutic index, so they have to be administered under a strict supervision. The proximity between effective and toxic doses is the cause of severe adverse effects to appear. Na⁺, K⁺-ATPase inhibition at therapeutic doses is the cause of their positive inotropic effect, since only little changes in [Na⁺]i are required for a large effect on contractile force (Lee 1985). Apart from this activity, they can act on other physiological systems, leading to adverse effects (Gillis 1986).

Cardiac glycosides also have well known antiproliferative effects on tumor cells (Shjratori 1967; Repke 1988; Repke 1995). Some cardiac glycosides have been evaluated in short term animal models. The conclusion from these experiments is that very high doses, probably toxic, would be needed for obtaining anticancer effects in humans (Cassady 1980). In contrast, recently it has been found that non-toxic concentrations of digitoxin and digoxin inhibits growth and induce apoptosis in different human malignant cell lines, whereas highly proliferating normal cells were not affected (Haux 1999 & 2000). The capability of cardiac glycosides to induce apoptosis has recently been confirmed in other studies (Kawazoe 1999). There is a great difference in susceptibility for cardiac glycosides in different species indicating that one can not extrapolate the results from animal models into humans (Repke 1988).

In vitro experiments the apoptosis-inducing effect was more potent for digitoxin than for digoxin, and for digitoxin there was a dose response pattern; the higher concentration the more apoptosis. Another recent report on the anticancer effects of different cardiac glycosides on tumor cell lines also confirms that digitoxin seems more potent than digoxin (Johansson 2001).

It has been shown that cardiac glycosides Oleandrin, Ouabain, and Digoxin induce apoptosis in androgen-independent human prostate cancer cell lines in vitro. Cell death was associated with early release of cytochrome c from mitochondria, followed by proteolytic processing of caspases 8 and 3. Oleandrin also promoted caspase activation, detected by cleavage poly (ADP-ribose) polymerase and hydrolysis of a peptide substrate (DEVD-pNA). Comparison of the rates of apoptosis in poorly metastatic PC3 M-Pro4 and highly metastatic PC3 M-LN4 subclones demonstrated that cell death was delayed in the latter because of a delay in mitochondrial cytochrome c release. Single-cell imaging of intracellular Ca(2+) fluxes demonstrated that the proapoptotic effects of the cardiac glycosides were linked to their abilities to induce sustained Ca(2+) increases in the cells. These results show that cardiac glycosides can be used to the treatment of metastatic prostate cancer. (McConkey 2000).

Further it is known that in vitro, cardiac glycosides may inhibit fibroblast growth factor-2 (FGF-2) export through membrane interaction with the Na⁺, K⁺-ATPase pump (Yeh 2001). It has been shown that Oleandrin (0.1 ng/mL) produced a 45.7% inhibition of FGF-2 release from PC3 cells and a 49.9% inhibition from DU145 cells. The water extract of the oleander plant (100 ng/mL) produced a 51.9 and 30.8% inhibition of FGF-2 release, respectively, in the two cell lines. These results demonstrate that the water extract, like Oleandrin, inhibited FGF-2 export in vitro from PC3 and DU145 prostate cancer cells in a concentration- and time-dependent fashion and may, therefore, contribute to the antitumor activity of the treatment for cancer (Smith 2001).

U.S. Pat. No. 6,071,885 claims cardiac glycosides, specifically, digoxin and ouabain, for the treatment of FGF-mediated pathophysiological condition in a patient. The pathophysiological condition is selected from melanoma, ovarian carcinoma, teratocarcinoma and neuroblastoma. However, the patent does not address the Na⁺, K⁺-ATPase inhibiting properties of these glycosides which are indirectly responsible for the FGF export inhibition (Yeh 2001). For example, Stewart et al (2000) and Grimes et al (1995) discusses the importance of the pump inhibition of these glycosides in prostate cancer cell lines. U.S. Pat. No. 6,281,197 similarly claims cardiac glycosides, especially digoxin and ouabain, for the treatment of complaications of diabetes involving the inhibition of the export of leaderless FGF proteins. However, a literature search on the internet using PUBMED site for cardiac glycoside and diabetes produced more than 300 publications and all of these publications imply the importance of Na⁺, K⁺-ATPase in diabetes mellitus. It has been shown that streptozotocin-induced diabetes mellitus in the rat is associated with a substantial increase in ouabain-sensitive ATPase activity along most of the nephron (Wald 1986). Further, it has been found that there is decrease in Na+-K+ pump concentration in nerve cells in diabetic rats and the decrease may be due to atrophy of the axons. In skeletal muscles, myocardium, and peripheral nerves, the observed decrease in Na+-K+ pump concentration may be important for the pathophysiology of diabetes (Kjeldsen 1987). Diabetic neuropathy is a degenerative complication of diabetes accompanied by an alteration of nerve conduction velocity (NCV) and Na⁺, K⁺-ATPase activity. Na⁺, K⁺-ATPase activity was significantly lower in sciatic nerve membranes of diabetic rats and significantly restored in diabetic animals that received fish oil supplementation. Diabetes induced a specific decrease of alpha1- and alpha3-isoform activity and protein expression in sciatic nerve membranes(Gerbi 1998). It has been observed that high glucose with suppressed Na+/K+ pump activity might induce an increase of Ca2+ influx through either Ca2+ channels or reverse Na+/Ca2+-exchange, possibly leading to the elevation of Ca2+-activated voltage-dependent K+ channels. Both a decrease in inward Na+ current and an increase in K+ conductance may result in decreased nerve conduction. In addition, a possible increase of axoplasmic Ca2+ concentration may lead to axonal degeneration.

These results provide a clue for understanding the pathophysiologic mechanism of diabetic neuropathy (Takigawa 2000).

Further it has been reported that there is a reduction in activity of the ouabain-sensitive Na⁺, K⁺-ATPase pump and a reduction in membrane permeability on the diabetic erythrocyte which is most marked in Type 1 diabetics (Jennings 1986). Further it has been found that the Na+-pumping activity, estimated from both Na⁺, K⁺-ATPase and ouabain binding, was significantly decreased in IDDM and NIDDM subjects, but its insulin sensitivity was retained only in young IDDM subjects (Baldini 1989). It has been observed that VSMC grown in high glucose concentration milieu manifests a decreased Na—K, and Ca transport in conjunction with an increase in intracellular concentration of Na and [Ca]i. These results suggest that high glucose, per se, may alter membrane permeability to cations, possibly leading to changes in VSMC contractility and/or proliferation. This abnormality seen in the diabetic state may closely link to the pathogenesis of diabetic angiopathy, thus as a result risking hypertension and vascular disease (Kuriyama 1994). Sennoune et al (2000) studied in rats the effect of streptozotocin-induced diabetes on liver Na⁺, K⁺-ATPase. Diabetes mellitus induced an increased Na⁺, K⁺-ATPase activity and an enhanced expression of the betal subunit; Diabetes mellitus led to a decrease in membrane fluidity and a change in membrane lipid composition. The results suggest that the increase of Na⁺, K⁺-ATPase activity can be associated with the enhanced expression of the beta1 subunit in the diabetic state, but cannot be attributed to changes in membrane fluidity as typically this enzyme will increase in response to an enhancement of membrane fluidity.

Further, the level of Na⁺, K⁺-ATPase activity and the number of enzyme units were about 30% lower in the red blood cells of diabetic patients than in healthy Caucasian controls (Raccah 1996).

The adenosine triphosphate-binding site, investigated by anisotropy decay studies of the fluorescent probe pyrene isothiocyanate, was modified in women with IDDM and it appears that the Na⁺, K⁺-ATPase of human placenta is altered in its disposition in IDDM (Zolese 1997). The alterations in small intestinal Na⁺, K⁺-ATPase expression in the chronic diabetic state appear to involve alterations in transcriptional and posttranscriptional events and may likely represent an adaptive response that leads to increased Na+-coupled monosaccharide absorption in the context of a perceived state of nutrient depletion (Wild 1999).

U.S. Pat. No. 5,872,103 describes a method for the prevention of mammary tumors by the administration of cardiac glycoside, especially, digoxin and digitoxin.

Further agents that can suppress the activation of nuclear factor-κB (NF-κB) and activator protein-1 (AP-1) may be able to block tumorigenesis and inflammation. Oleandrin blocked tumor necrosis factor (TNF)-induced activation of NF-κB in a concentration- and time-dependent manner. This effect was mediated through inhibition of phosphorylation and degradation of IκBα, an inhibitor of NF-κB. The water extract of oleander also blocked TNF-induced NF-κB activation; subsequent fractionation of the extract revealed that this activity was attributable to Oleandrin. The effects of Oleandrin were not cell type specific, because it blocked TNF-induced NF-κB activation in a variety of cells. NF-κB-dependent reporter gene transcription activated by TNF was also suppressed by Oleandrin. The TNF-induced NF-κB activation cascade involving TNF receptor 1/TNF receptor-associated death domain/TNF receptor-associated factor 2/NF-κB-inducing kinase/IκBα kinase was interrupted at the TNF receptor-associated factor 2 and NF-κB-inducing kinase sites by Oleandrin, thus suppressing NF-κB reporter gene expression. Oleandrin blocked NF-κB activation induced by phorbol ester and lipopolysaccharide. Oleandrin also blocked AP-1 activation induced by TNF and other agents and inhibited the TNF-induced activation of c-Jun NH2terminal kinase. Overall, these results indicate that Oleandrin inhibits activation of NF-κB and AP-1 and their associated kinases. These results may provide a molecular basis for the ability of Oleandrin to suppress inflammation and perhaps tumorigenesis. (Manna 2000)

While the water extract of the Nerium Oleander leaves has shown to ameliorate the cell proliferative diseases in humans, it is rather difficult to develop the extract as a parenteral pharmaceutical product suitable for commercialization due to the presence of several compounds. Since the anti-tumor activity of the oleander extract has been shown to be due to the presence of Oleandrin and oleandrogenin in the extract it is desirable to develop Oleandrin as an anti-tumor agent. The term cell-proliferative diseases is meant here to denote malignant as well as non-malignant cell populations which often appear morphologically to differ from the surrounding tissue.

As described before, Oleandrin is extremely toxic due to its cardiac properties and it is believed that the non-toxic nature of the water extract is due to the encapsulation of the water insoluble Oleandrin and oleandrogenin molecules into the polysaccarides present in the extract. The encapsulated Oleandrin and oleandrogenin is soluble in water and Oleandrin is released slowly upon administration through injection. Also, the amount of Oleandrin encapsulated by the extraction procedure is very small (2-5 microgram per mg) and it should be possible to develop alternate delivery vehicles to reduce the toxicity of Oleandrin and thereby increase its therapeutic value. It is highly desirable to develop new procedures for the increase of the therapeutic value of Oleandrin to treat cancers such as metastatic prostate cancer.

There are many potential barriers to the effective delivery of a toxic drug in its active form to solid tumors. Most small-molecule chemotherapeutic agents have a large volume of distribution on i.v. administration. The result of this is often a narrow therapeutic index due to a high level of toxicity in healthy tissues. Through encapsulation of drugs in a macromolecular carrier, such as a liposome, the volume of distribution is significantly reduced and the concentration of drug in the tumor is increased. This results in a decrease in the amount and types of nonspecific toxicities and an increase in the amount of drug that can be effectively delivered to the. Under optimal conditions, the drug is carried within the liposomal aqueous space while in the circulation but leaks at a sufficient rate to become bioavailable on arrival at the tumor. The liposome protects the drug from metabolism and inactivation in the plasma, and due to size limitations in the transport of large molecules or carriers across healthy endothelium, the drug accumulates to a reduced extent in healthy tissues. However, discontinuities in the endothelium of the tumor vasculature have been shown to result in an increased extravasation of large carriers and, in combination with an impaired lymphatics, an increased accumulation of liposomal drug at the tumor. All of these factors have contributed to the increased therapeutic index observed with liposomal formulations of some chemotherapeutic agents (Drummond et al 1999).

Protein microspheres have also been reported in the literature as carriers of pharmacological or diagnostic agents. Microspheres of albumin have been prepared by either heat denaturation or chemical crosslinking. Heat denatured microspheres are produced from an emulsified mixture (e.g., albumin, the agent to be incorporated, and a suitable oil) at temperatures between 100° C. and 150° C. The microspheres are then washed with a suitable solvent and stored. Leucuta et al. (1988) describe the method of preparation of heat denatured microspheres. The procedure for preparing chemically crosslinked microspheres involves treating the emulsion with glutaraldehyde to crosslink the protein, followed by washing and storage. Lee et al., (1981) and U.S. Pat. No. 4,671,954 teach this method of preparation. The above techniques for the preparation of protein microspheres as carriers of pharmacologically active agents, although suitable for the delivery of water-soluble agents, are incapable of entrapping water-insoluble ones. This limitation is inherent in the technique of preparation which relies on crosslinking or heat denaturation of the protein component in the aqueous phase of a water-in-oil emulsion. Any aqueous-soluble agent dissolved in the protein-containing aqueous phase may be entrapped within the resultant crosslinked or heat-denatured protein matrix, but a poorly aqueous-soluble or oil-soluble agent cannot be incorporated into a protein matrix formed by these techniques.

U.S. Pat. Nos. 5439686 and 5916596 teach the methods for the production of particulate vehicles for the intravenous administration of pharmacologically active agents. They disclose methods for the in vivo delivery of substantially water insoluble anticancer drug taxol. The suspended particles are encased in a polymeric shell formulated from a biocompatible polymer, and have a diameter of less than about 1 micron. The polymeric shell contains particles of taxol, and optionally a biocompatible dispersing agent in which pharmacologically active agent can be either dissolved or suspended.

Liposomes are phospholipid vesicles, composing mainly naturally occurring substances that are nontoxic and biodegradable (Lasic 1993) and are made up of at least one lipid bilayer membrane containing an entrapped aqueous internal compartment. When combined with water, phospholipids immediately form a sphere because one end of each molecule is water soluble, while the opposite end is water insoluble. Water-soluble medications added to the water are trapped inside the aggregation of the hydrophobic ends; fat-soluble medications are incorporated into the phospholipid layer.

Vesiculation of natural phospholipid bilayer is not a spontaneous process. Physical and chemical methods are used to produce well-defined liposomes from hydrated lipids. Since the discovery by Bangham in 1961, many processing methods for liposome production have been developed. The majority of these methods require the input of high energy (e.g., ultrasonic treatment, high pressure, and/or elevated temperatures) to disperse low critical micelle concentration phospholipids as a metastable liposome phase (Lasic and Paphadjopoulos 1998).

Liposomes have been employed for a number of therapeutic applications, in particular, for delivering drugs to target cells following systemic administration (Drummond et al. 1999; Gibbson and Paphadjopoulos 1988; Lasic and Paphadjopoulos 1998; Olson et al. 1982; Rahman et al. 1982). Liposomal formulations of pharmaceutical agents are superior to drugs injected in the free form. When used in the delivery of certain cancer drugs, liposomes help to shield healthy cells from the drugs' toxicity and prevent their concentration in vulnerable tissues (e.g., the kidneys, and liver), lessening or eliminating the common side effects of nausea, fatigue, and hair loss. For instance, liposomal formulations of the anticancer agent vincristine exhibit greater efficacy against L1210 leukemia cells than does free vincristine and have reduced collateral toxicity. Liposomes have also been used to deliver certain vaccines, enzymes, or insulin to the body. They have also been used experimentally to carry normal genes into a cell in order to replace defective, disease-causing genes.

Commercial liposomal drug delivery is gaining attention because of the enhanced stability of the liposomes, reduced toxicity, sustained-drug release, enhanced blood circulation time, and increased accumulation of liposomes in the target sites. Reduction in toxicity may result from the ability of liposomes to decrease drug exposure, and subsequent damage, to susceptible tissues (Allen et al. 1991). In fact, the first liposomal drug oncology drug approved for medicinal use, in liposomal form, are of the anthracyclines daunorubicin (DaunoXome; Nextstar Pharmaceuticals, Boulder, CO), EVACET (The Liposome Company, Inc.) and DOX [Doxil; Alza Corporation, Palo Alto, Calif. (CAELYX in Europe)].

Phospholipid in its simplest form is composed of glycerol bonded to two fatty acids and a phosphate group. The resulting compound called phosphatidic acid contains a region (the fatty acid component) that is fat-soluble along with a region (the charged phosphate group) that is water-soluble. Most phospholipids also have an additional chemical group bound to the phosphate. For example, it may be connected with choline; the resulting phospholipid is called phosphatidylcholine, or lecithin. Other phospholipids include phosphatidylglycerol, phosphatidylinositol, phosphatidylserine, and phosphatidylethanolamine. The fat-soluble portions associate with the fat-soluble portions of other phospholipids while the water-soluble regions remain exposed to the surrounding solvent. The phospholipids of the cell membrane form into a sheet two molecules thick with the fat-soluble portions inside shielded on both sides by the water-soluble portions. This stable structure provides the cell membrane with its integrity.

Liposomes consist of amphipathic lipid molecules, with phospholipids being the major component. Most commonly, phosphatidylcholine is used as the primary constituent. Other lipids, including phosphatidylethanolamine, phosphatidylserine, sphingomyelin, glycolipids and sterols are often added. The physical characteristics of liposomes depend on pH, ionic strength and phase transition temperatures. The phase transition consists of a closely packed, ordered structure, called as the gel-state, to a loosely packed, lessordered structure, known as the fluid state. The phase transition temperature (T_{c}) depends on the acyl chain length, degree of saturation, and polar head group. For example, the T_{c} of egg phosphatidylcholine with a high degree of unsaturation of the acyl chains and varying chain length is −15° C. However, in a fully saturated distearoylphosphatidylcholine (DSPC), T_{c} is over 50° C. Most liposomal formulations contain cholesterol in order to form a more closely packed bilayer system during preparation. Cholesterol addition to phosphatidylcholine changes the melting behavior of the bilayer, as cholesterol tends to eliminate the phase transition. Cholesterol addition has a condensing effect on the fluid-state bilayer and strongly reduces bilayer permeability.

Biologically active drug molecules can be trapped either within the aqueous compartment or incorporated within the bilayer themselves depending on their hydrophilicity or lipophilicity. However, in many instances, the drug leaks from the liposomes during long-term storage, lyophilizaion and reconstitution. Protecting liposomes by polymerized protein molecules minimizes or eliminates the drug leakage from the liposome. Furthermore, as the protein uniformly coats or forms a shell around the liposome, thereby the drug is protected from any further degradation from the liposome. This method also allows for the entrapment of a high concentration of drug inside the liposome compartment.

Major advances in improving the therapeutic index of amphotericin B encapsulated in liposomes have been demonstrated in counteracting systemic fungal infections in cancer patients (Olsen et al. 1982). The liposomal entrapment of this antifungal drug causes a remarkable reduction in toxicity. Liposomes have also been found to be effective in delivering doxorubicin (Williams et al. 1993), vincristine (Woodle et al. 1992), vinblastine, actinomycin-D, arabinoside, cytosine, daunomycin (Julliano and Stamp 1978), mitoxantrone, epirubicin, daunorubicin, (Madden et al 1990) and paclitaxel (Suffness 1995). In a liposomal drug delivery system, if the drug is highly hydrophobic, it tends to associate mainly with the bilayer compartment (Sharmaetal., 1995, 1997).

However, following i.v. administration, on some occasion, the premature release and leakage of the drug from the liposome result in faster distribution of the drug in the plasma component, higher toxicity and less amount of the drug released at the tumor site. Furthermore, for pegylated liposomal doxorubicin, a novel dose-limiting form of skin toxicity known as palmar-plantar erythrodysaesthesia or hand-foot syndrome has been described (Gordon et al. 1995). This problem can be overcome by the improvement in the design of the drug carrier, by uniform coating of the protein onto the liposome. This type of coating by protein molecules on the liposome is referred here as protein stabilized liposomes (PSL).

Following parenteral administration, a drug entrapped in the PSL nanoparticles is protected from premature release and immediate dilution or degradation. PSL nanoparticles can alter the pharmacokinetics and biodistribution. This can reduce toxic side effects and increase efficacy of the therapy. The PSL nanoparticles are distributed within the body much differently than free drugs. When administered intravenously to healthy animals and humans, most of the drug from the nanoparticles accumulates in the liver, spleen, lungs, bone marrow and lymph nodes. These nanoparticles also accumulate at sites of inflammation and infection and in some solid tumors.

PSL nanoparticle formulations, with protein stabilized liposomes have a reduced uptake by the RES, and, consequently, show a longer circulation time, increased biological and chemical stability, and increased accumulation in tumor-sites. The PSL nanoparticle formulations with components such as phosphatidyl insitol, monosialogangolioside, spingosomes, poly(ethylene glycol)-derivatized distearoylphosphatidylethanolamine (PEG-DSPE), poly(ethylene glycol)-derivatized ceramides (PEG-CER) show prolonged circulation time in blood. Most importantly, PSL nanoparticle formulations produce a marked enhancement of anti-tumor activity in mice against different carcinomas with a concomitant decrease in toxicity.

The PSL nanoparticle formulation protects the drug from metabolism and inactivation in the plasma, and due to size limitations in the transport of large molecules or carriers across healthy endothelium, the drug accumulates to a reduced extent in healthy tissues (Working et al., 1994; Mayer et al., 1989). However, discontinuities in the endothelium of vasculature provide an increased accumulation of PSL nanoparticles at the tumor. These PSL nanoparticles have sizes below 400 nm, preferably below 200 nm, and more preferably below 120 nm having hydrophilic proteins coated onto the surface of the nanoparticles.

### SUMMARY OF THE INVENTION

The present invention relates to the liposomal formulation of digitalis glycosides. In particular embodiments, the invention relates to the use of the digitalis glycosides, as anti-tumor agents. The inventors have demonstrated that the liposomal formulations of the digitalis glycosides disclosed herein, for example, exerts cytotoxic effects in human cancer cell lines and in animals transplanted with these cancer cells.

The present invention is also directed to the use of PSL nanoparticle formulations containing digitalis glycosides in the treatment of cell-proliferate disease cancer in humans. More particularly, the invention is directed to the use of novel PSL nanoparticle formulations with reduced toxicity, long-circulating time in the bloodstream and able to deliver the drug to the target sites such as tumor sites. These PSL nanoparticle formulations are higher than or substantially equivalent in efficacy to each one of the pharmaceutical agents in its free form, yet generally have low toxicity. The pharmaceutical compositions of PSL nanoparticle formulations comprising a mixture of egg phosphatidylcholine (EPC), hydrogenated soy phosphatidylcholine (HSPC), phosphatidylethanolamine (PE), phosphatidylglycerol(PG), phosphatidylinsitol (PI), monosialogangolioside and spingomyelin (SPM); the derivatized vesicle forming lipids such as poly(ethylene glycol)-derivatized distearoylphosphatidylethanolamine (PEG-DSPE), poly(ethylene glycol)-derivatized ceramides (PEG-CER), distearoylphosphatidylcholine (DSPC), dimyristoyl-phosphatidylcholine (DMPC), dimyristoylphosphatidylglycerol (DMPG), and dipalmitoylphosphatidylcholine (DPPC), cholesterol, and proteins.

In a preferred embodiment the liposomal or PSL composition comprises at least one digitalis glycosides. It will, of course, be understood that the composition may further comprise a second digitalis glycosides, or one or more other pharmacologically-active compounds, and particularly one or more anti-tumor compounds. The methods of the invention may thus entail the administration of one, two, three, or more, of digitalis glycosides. The maximum number of species that may be administered is limited only by practical considerations, such as the particular effects of each compound.

The present invention also provides the preparation and use of a variety of PSL nanoparticle formulations. In another aspect, the present invenstion provides novel PSL nanoparticle formulations of digitalis glycosides with reduced toxicity, high drug to lipid ratio, long-circulating time in the bloodstream and able to deliver the drug to the target sites, including tumor sites. In another aspect, the present invention provides an effective method to reduce the growth of cancers or reducing the incidence of metastases.

In yet another aspect, the present invention provides an effective method for treating diseases such as anti-inflammation, cancer and arthritis in a warm-blooded animal.

In yet another embodiment, a PSL nanoparticle formulation is prepared by solvent evaporation of an oil-in-water emulsion consisting of an digitalis glycosides, cholesterol, protein, and lipids. A homogenozer or a microfluidizer with a pressure in the range of about 3000 to 40,000 psi or a sonicator and an evaporator are used to prepare the fine emulsion and nanoparticles. Lipophilic therapeutic compounds are dissolved in the oil phase.

This invention also provides a method for producing PSL nanoparticles having size less than 220 nm, preferably 10-220 nm and most preferably between about 30-220 nm. These PSL nanoparticles can be sterile filtered through a 0.22 μm filter.

In yet another embodiment of the method, the sterile-filtered PSL nanoparticles can be lyophilized in the form of a cake in vials using cryoprotectants such as sucrose, mannitol, trehalose or the like. The lyophized cake can be reconstituted to the original liposomes, without modifying the particle size of the PSL nanoparticles. These nanoparticles are administered by a variety of routes, preferably by intravenous, parenteral, intratumoral and oral or routes. The invention also includes a method of treating cancer with digitalis glycosides. This method comprises administration of an effective amount of a suitable liposomal composition or PSL formulation containing the digitalis glycosides to a subject in need thereof. Administration is preferably by either intramuscular or intravenous injections. The treatment may be maintained as long as necessary and may be used in conjunction with other forms of treatment.

It is a further object of the present invention to deliver the highly toxic compound Oleandrin and other digitalis glycosides in a composition of microparticles or nanoparticles, optionally suspended in a suitable biocompatible liquid.

It is yet another object of the present invention to provide a method for the formation of submicron particles (nanoparticles) of digitalis glycosides by a solvent evaporation technique from an oil-in-water emulsion using proteins as stabilizing agents in the absence of any conventional surfactants, and in the absence of any polymeric core material.

In accordance with the present invention, we have discovered that digitalis glycosides can be delivered in the form of microparticles or nanoparticles that are suitable for parenteral administration in aqueous suspension. This mode of delivery obviates the necessity for administration of substantially water insoluble compound Oleandrin through the aqueous extract formulation as described in U.S. patent.

Thus, in accordance with the present invention, there are provided methods for the formation of nanoparticles of digitalis glycosides by a solvent evaporation technique from an oil-in-water emulsion prepared under conditions of high shear forces (e.g., sonication, high pressure homogenization, or the like) without the use of any conventional surfactants, and without the use of any polymeric core material to form the matrix of the nanoparticle. Instead, proteins (e.g., human serum albumin) are employed as a stabilizing agent.

The invention further provides a method for the reproducible formation of unusually small nanoparticles (less than 200 nm diameter), which can be sterile-filtered through a 0.22 micron filter. This is achieved by addition of a water soluble solvent (e.g. ethanol) to the organic phase and by carefully selecting the type of organic phase, the phase fraction and the drug concentration in the organic phase. The ability to form nanoparticles of a size that is filterable by 0.22 micron filters is of great importance and significance, since formulations which contain a significant amount of any protein (e.g., albumin), cannot be sterilized by conventional methods such as autoclaving, due to the heat coagulation of the protein.

In accordance with another embodiment of the present invention, we have developed compositions useful for in vivo delivery of substantially water insoluble digitalis glycosides. Invention compositions comprise substantially water insoluble digitalis glycosides (as a solid or liquid) contained within a polymeric shell. The polymeric shell is a crosslinked biocompatible polymer. The polymeric shell, containing substantially water insoluble pharmacologically active agents therein, can then be suspended in a biocompatible aqueous liquid for administration.

The invention further provides a drug delivery system in which part of the molecules of digitalis glycosides are bound to the protein (e.g., human serum albumin), and are therefore immediately bioavailable upon administration to a mammal. The other portion of the pharmacologically active agent is contained within nanoparticles coated by protein. The nanoparticles containing the pharmacologically active agent are present as a pure active component, without dilution by any polymeric matrix.

In accordance with the present invention, there are also provided submicron particles in powder form, which can easily be reconstituted in water or saline. The powder is obtained after removal of water by lyophilization. Human serum albumin serves as the structural component of invention nanoparticles, and also as a cryoprotectant and reconstitution aid. The preparation of particles filterable through a 0.22 micron filter according to the invention method as described herein, followed by drying or lyophilization, produces a sterile solid formulation useful for intravenous injection.

The invention provides, in a particular aspect, a composition of anti-cancer drug Oleandrin in the form of nanoparticles in a liquid dispersion or as a solid which can be easily reconstituted for administration. While it is recognized that particles produced according to the invention can be either crystalline, amorphous, or a mixture thereof, it is generally preferred that the drug be present in the formulation in an amorphous form. This would lead to greater ease of dissolution and absorption, resulting in better bioavailability.

### DETAILED DESCRIPTION OF THE INVENTION

It is understood as “digitalis activity” the ability to inhibit Na⁺, K⁺-ATPase through acting onto the digitalis receptor, along with the ability to display a positive inotropic effect. Such an action is performed by several natural, semisynthetic and synthetic compounds (Thomas 1992). Among the natural compounds, there are three groups: steroidal butenolides and pentadienolides, known as “cardiotonic steroids” or “digitalic compounds” and Erythrophleum alkaloids. The word “digitalis” is often used as a generic word for all cardiotonic steroids; similarly, the receptor for these compounds is generally known as “digitalis receptor”. Digitalis glycosides or also called as digitalis-type glycosides or also called as cardiac glycosides are compounds bearing a steroidal genin or aglycone with one or several sugar molecules attached to position C-3. In the case of toad venom, sugar is replaced by suberylarginine.

As used herein, the term “micron” refers to a unit of measure of one one-thousandth of a millimeter.

As used herein, the term “nm” or the term “nanometers” refers to a unit of measure of one one-billionth of a meter.

As used herein, the term “biocompatible” describes a substance that does not appreciably alter or affect in any adverse way, the biological system into which it is introduced.

As used herein, the term “substantially water insoluble pharmaceutical agent” means biologically active chemical compounds which are poorly soluble or almost insoluble in water. Examples of such compounds are paclitaxel, oleandrin, cyclosporine, digitoxin and the like.

As used herein, the term “cell-proliferative diseases” is meant here to denote malignant as well as non-malignant cell populations which often appear morphologically to differ from the surrounding tissue.

As discussed above, the present invention provides liposomal and PSL nanoparticle formulations of digitalis glycosides and methods of preparing and employing such formulations. The advantages of these PSL nanoparticle formulations are that a drug is entrapped in either dissolved or precipitated form. These compositions have been observed to provide a very low toxicity form of the pharmacologically active agent that can be delivered in the form of nanoparticles or suspensions by slow infusions or by bolus injection or by other parenteral or oral delivery routes. These PSL nanoparticles have sizes below 400 nm, preferably below 200 nm, and more preferably below 120 nm having hydrophilic proteins coated onto the surface of the nanoparticles.

The vesicle forming lipids such as, egg phosphatidylcholine (EPC), hydrogenated soy phosphatidylcholine (HSPC), phosphatidylethanolamine (PE), phosphatidylglycerol(PG), phosphatidylinsitol (PI), monosialogangolioside and spingomyelin (SPM); the derivatized vesicle forming lipids such as poly(ethylene glycol)-derivatized distearoylphosphatidylethanolamine (PEG-DSPE) and poly(ethylene glycol)-derivatized cerarmides (PEG-CER); and cholesterol are dissolved in organic solvents along with one or more digitalis glycoside. The phospholipids can be either synthetic or derived from natural sources such as egg or soy. The phospholipids can be distearoylphosphatidylcholine (DSPC), dimyristoylphosphatidylcholine (DMPC), dimyristoylphosphatidylglycerol (DMPG), and dipalmitoylphosphatidylcholine (DPPC).

These lipids are dissolved in the organic solvent along with the digitalis glycoside, and the protein is dissolved in the aqueous phase. The organic phase is added to the aqueous phase and subjected to high shear stress. This results in a fine oil-in-water emulsion. Evaporation of the solvent from the emulsion leads to the formation PSL nanoparticles with a high digitalis glycoside to lipid-protein ratio (wt/wt). The drug to lipid-protein weight ratio varies between 0.01 and 1, preferably between 0.05 and 1.

In order to make the protein stabilized liposomal nanoparticles, digitalis glycoside, lipid and other agents are dissolved in a suitable solvent (e.g., chloroform, methylene chloride, ethyl acetate, ethanol, tetrahydrofuran, dioxane, acetonitrile, acetone, dimethyl sulfoxide, dimethyl formamide, methyl pyrrolidinone, or the like, as well as mixtures of any two or more thereof). Additional solvents contemplated for use in the practice of the present invention include soybean oil, coconut oil, olive oil, safflower oil, cotton seed oil, sesame oil, orange oil, limonene oil, C1-C20 alcohols, C2-C20 esters, C3-C20 ketones, polyethylene glycols, aliphatic hydrocarbons, aromatic hydrocarbons, halogenated hydrocarbons and combinations thereof.

In the next stage, in order to make the protein stabilized liposomal nanoparticles, a protein (e.g., human serum albumin) is added (into the aqueous phase) to act as a stabilizing agent for the formation of stable nanodroplets. Protein is added at a concentration in the range of about 0.05 to 25% (w/v), more preferably in the range of about 0.5%-5% (w/v).

In the next stage, in order to make the protein stabilized liposomal nanoparticles, an emulsion is formed by homogenization under high pressure and high shear forces. Such homogenization is conveniently carried out in a high pressure homogenizer, typically operated at pressures in the range of about 3,000 up to 30,000 psi. Preferably, such processes are carried out at pressures in the range of about 6,000 up to 25,000 psi. The resulting emulsion comprises very small nanodroplets of the nonaqueous solvent containing the digitalis glycoside, lipid and other agents. Acceptable methods of homogenization include processes imparting high shear and cavitation such as high pressure homogenization, high shear mixers, sonication, high shear impellers, and the like.

Finally, in order to make the protein stabilized liposomal nanoparticles, the solvent is evaporated under reduced pressure to yield a colloidal system composed of protein stabilized liposomal nanoparticles of digitalis glycoside in liposome and protein. Acceptable methods of evaporation include the use of rotary evaporators, falling film evaporators, spray driers, freeze driers, and the like. Following evaporation of solvent, the liquid suspension may be dried to obtain a powder containing the pharmacologically active agent and protein. The resulting powder can be redispersed at any convenient time into a suitable aqueous medium such as saline, buffered saline, water, buffered aqueous media, solutions of amino acids, solutions of vitamins, solutions of carbohydrates, or the like, as well as combinations of any two or more thereof, to obtain a suspension that can be administered to mammals. Methods contemplated for obtaining this powder include freeze-drying, spray drying, and the like.

In accordance with a specific embodiment of the present invention, there is provided a method for the formation of unusually small submicron liposomal particles containing digitalis gfycoside, i.e., particles which are less than 200 nanometers in diameter. Such particles are capable of being sterile-filtered before use in the form of a liquid suspension. The ability to sterile-filter the end product of the invention formulation process (i.e., the drug particles) is of great importance since it is impossible to sterilize dispersions which contain high concentrations of protein (e.g., serum albumin) by conventional means such as autoclaving.

In order to obtain sterile-filterable protein stabilized liposomal particles of digitalis glycosides (i.e., particles<200 nm), the digitalis glycoside, lipids and other agents are initially dissolved in a substantially water immiscible organic solvent (e.g., a solvent having less than about 5% solubility in water, such as, for example, chloroform) at high concentration, thereby forming an oil phase containing the digitalis glycoside, lipids and other agents. Suitable solvents are set forth above. Next, a water miscible organic solvent (e.g., a solvent having greater than about 10% solubility in water, such as, for example, ethanol) is added to the oil phase at a final concentration in the range of about 1%-99% v/v, more preferably in the range of about 5%-25% v/v of the total organic phase. The water miscible organic solvent can be selected from such solvents as ethyl acetate, ethanol, tetrahydrofuran, dioxane, acetonitrile, acetone, dimethyl sulfoxide, dimethyl formamide, methyl pyrrolidinone, and the like. Alternatively, the mixture of water immiscible solvent with the water miscible solvent is prepared first, followed by dissolution of the digitalis glycoside, lipids and other agents in the mixture.

In the next stage, in order to make the protein stabilized liposomal nanoparticles of digitalis glycosides, human serum albumin or any other suitable stabilizing agent as described above is dissolved in aqueous media. This component acts as a stabilizing agent for the formation of stable nanodroplets. Optionally, a sufficient amount of the first organic solvent (e.g. chloroform) is dissolved in the aqueous phase to bring it close to the saturation concentration. A separate, measured amount of the organic phase (which now contains the digitalis glycosides, the first organic solvent and the second organic solvent) is added to the saturated aqueous phase, so that the phase fraction of the organic phase is between about 0.5%-015% v/v, and more preferably between 1% and 8% v/v. Next, a mixture composed of micro and nanodroplets is formed by homogenization at low shear forces. This can be accomplished in a variety of ways, as can readily be identified by those of skill in the art, employing, for example, a conventional laboratory homogenizer operated in the range of about 2,000 up to about 15,000 rpm. This is followed by homogenization under high pressure (i.e., in the range of about 3,000 up to 30,000 psi). The resulting mixture comprises an aqueous protein solution (e.g., human serum albumin), the digitalis glycoside, lipids, other agents, the first solvent and the second solvent. Finally, solvent is rapidly evaporated under vacuum to yield a colloidal dispersion system (liposomal digitalis glycoside and protein) in the form of extremely small nanoparticles (i.e., particles in the range of about 50 nm-200 nm diameter), and thus can be sterile-filtered. The preferred size range of the particles is between about 50 nm-170 nm, depending on the formulation and operational parameters.

The protein stabilized liposomal nanoparticles prepared in accordance with the present invention may be further converted into powder form by removal of the water therefrom, e.g., by lyophilization at a suitable temperature-time profile. The protein (e.g., human serum albumin) itself acts as a cryoprotectant, and the powder is easily reconstituted by addition of water, saline or buffer, without the need to use such conventional cryoprotectants as mannitol, sucrose, glycine, and the like. While not required, it is of course understood that conventional cryoprotectants may be added to invention formulations if so desired. The liposomal shell containing digitalis glycoside allows for the delivery of high doses of the pharmacologically active agent in relatively small volumes.

According to this embodiment of the present invention, the liposome containing digitalis glycoside has a cross-sectional diameter of no greater than about 10 microns. A cross-sectional diameter of less than 5 microns is more preferred, while a cross-sectional diameter of less than 1 micron is presently the most preferred for the intravenous route of administration.

Proteins contemplated for use as stabilizing agents in accordance with the present invention include albumins (which contain 35 cysteine residues), immunoglobulins, caseins, insulins (which contain 6 cysteines), hemoglobins (which contain 6 cysteine residues per α2 β2 unit), lysozymes (which contain 8 cysteine residues), immunoglobulins, α-2-macroglobulin, fibronectins, vitronectins, fibrinogens, lipases, and the like. Proteins, peptides, enzymes, antibodies and combinations thereof, are general classes of stabilizers contemplated for use in the present invention. A presently preferred protein for use is albumin. Specific antibodies may also be utilized to target the nanoparticles to specific locations.

In the preparation of invention compositions, a wide variety of organic media can be employed to suspend or dissolve the substantially water insoluble digitalis glycosides. Organic media contemplated for use in the practice of the present invention include any nonaqueous liquid that is capable of suspending or dissolving the pharmacologically active agent, but does not chemically react with either the polymer employed to produce the shell, or the pharmacologically active agent itself. Examples include vegetable oils (e.g., soybean oil, olive oil, and the like), coconut oil, safflower oil, cotton seed oil, sesame oil, orange oil, limonene oil, aliphatic, cycloaliphatic, or aromatic hydrocarbons having 4-30 carbon atoms (e.g., n-dodecane, n-decane, n-hexane, cyclohexane, toluene, benzene, and the like), aliphatic or aromatic alcohols having 2-30 carbon atoms (e.g., octanol, and the like), aliphatic or aromatic esters having 2-30 carbon atoms (e.g., ethyl caprylate (octanoate), and the like), alkyl, aryl, or cyclic ethers having 2-30 carbon atoms (e.g., diethyl ether, tetrahydrofuran, and the like), alkyl or aryl halides having 1-30 carbon atoms (and optionally more than one halogen substituent, e.g., CH₃Cl, CH₂Cl₂, CH₂Cl—CH₂Cl, and the like), ketones having 3-30 carbon atoms (e.g., acetone, methyl ethyl ketone, and the like), polyalkylene glycols (e.g., polyethylene glycol, and the like), or combinations of any two or more thereof.

Especially preferred combinations of organic media contemplated for use in the practice of the present invention typically have a boiling point of no greater than about 200° C., and include volatile liquids such as dichloromethane, chloroform, ethyl acetate, benzene, and the like (i.e., solvents that have a high degree of solubility for the pharmacologically active agent, and are soluble in the other organic medium employed), along with a higher molecular weight (less volatile) organic medium. When added to the other organic medium, these volatile additives help to drive the solubility of the pharmacologically active agent into the organic medium. This is desirable since this step is usually time consuming. Following dissolution, the volatile component may be removed by evaporation (optionally under vacuum).

The liposomes containing digitalis glycoside stabilized with protein, prepared as described above, are delivered as a suspension in a biocompatible aqueous liquid. This liquid may be selected from water, saline, a solution containing appropriate buffers, a solution containing nutritional agents such as amino acids, sugars, proteins, carbohydrates, vitamins or fat, and the like.

For increasing the long-term storage stability, the PSL nanoparticle formulations may be frozen and lyophilized in the presence of one or more protective agents such as sucrose, mannitol, trehalose or the like. Upon rehydration of the lyophilized PSL nanoparticle formulations, the suspension retains essentially all the drug previously loaded and the particle size. The rehydration is accomplished by simply adding purified or sterile water or 0.9% sodium chloride injection or 5% dextrose solution followed by gentle swirling of the suspension. The potency of the drug in a PSL nanoparticle formulation is not lost after lyophilization and reconstitution.

The PSL nanoparticle formulation of the present invention is shown to be less toxic than the drug administered in its free form. Determination of toxicity in mice has shown about 1- to 20-fold decrease in acute LD₅₀ values for PSL oleandrin nanoparticle formulations as compared to the free oleandrin. The LD₅₀ values are dependent on the lipid and protein compositions. Furthermore, PSL nanoparticle formulations containing Oleandrin exhibit 1 to 100-fold decrease in toxicity as compared to the drug in its free form. PSL nanoparticle formulations with low LD₅₀ values show low drug accumulation levels in heart, lung and kidney tissues. Although administration PSL nanoparticle formulations lead to their uptake by liver, acute liver damage is not observed.

In order to make the protein stabilized nanoparticles without the lipids, digitalis glycoside is dissolved in a suitable solvent (e.g., chloroform, methylene chloride, ethyl acetate, ethanol, tetrahydrofuran, dioxane, acetonitrile, acetone, dimethyl sulfoxide, dimethyl formamide, methyl pyrrolidinone, or the like, as well as mixtures of any two or more thereof). Additional solvents contemplated for use in the practice of the present invention include soybean oil, coconut oil, olive oil, safflower oil, cotton seed oil, sesame oil, orange oil, limonene oil, C1-C20 alcohols, C2-C20 esters, C3-C20 ketones, polyethylene glycols, aliphatic hydrocarbons, aromatic hydrocarbons, halogenated hydrocarbons and combinations thereof. Unlike conventional methods for nanoparticle formation, a polymer (e.g. polylactic acid) is not dissolved in the solvent. The oil phase employed in the preparation of invention compositions contains only the digitalis like molecules dissolved in solvent.

Next, in order to make the protein stabilized nanoparticles, a protein (e.g., human serum albumin) is added (into the aqueous phase) to act as a stabilizing agent for the formation of stable nanodroplets. Protein is added at a concentration in the range of about 0.05 to 25% (w/v), more preferably in the range of about 0.5%-5% (w/v). Unlike conventional methods for nanoparticle formation, no surfactant (e.g. sodium lauryl sulfate, lecithin, tween 80, pluronic F-68 and the like) is added to the mixture.

Next, in order to make the protein stabilized nanoparticles, an emulsion is formed by homogenization under high pressure and high shear forces. Such homogenization is conveniently carried out in a high pressure homogenizer, typically operated at pressures in the range of about 3,000 up to 30,000 psi. Preferably, such processes are carried out at pressures in the range of about 6,000 up to 25,000 psi. The resulting emulsion comprises very small nanodroplets of the nonaqueous solvent (containing the dissolved pharmacologically active agent) and very small nanodroplets of the protein stabilizing agent. Acceptable methods of homogenization include processes imparting high shear and cavitation such as high pressure homogenization, high shear mixers, sonication, high shear impellers, and the like.

Finally, in order to make the protein stabilized nanoparticles, the solvent is evaporated under reduced pressure to yield a colloidal system composed of protein stabilized nanoparticles of pharmacologically active agent and protein. Acceptable methods of evaporation include the use of rotary evaporators, falling film evaporators, spray driers, freeze driers, and the like. Following evaporation of solvent, the liquid suspension may be dried to obtain a powder containing the pharmacologically active agent and protein. The resulting powder can be redispersed at any convenient time into a suitable aqueous medium such as saline, buffered saline, water, buffered aqueous media, solutions of amino acids, solutions of vitamins, solutions of carbohydrates, or the like, as well as combinations of any two or more thereof, to obtain a suspension that can be administered to mammals. Methods contemplated for obtaining this powder include freeze-drying, spray drying, and the like.

In accordance with a specific embodiment of the present invention, there is provided a method for the formation of unusually small submicron particles of digitalis glycosides (nanoparticles), i.e., particles which are less than 200 nanometers in diameter. Such particles are capable of being sterile-filtered before use in the form of a liquid suspension. The ability to sterile-filter the end product of the invention formulation process (i.e., the drug particles) is of great importance since it is impossible to sterilize dispersions which contain high concentrations of protein (e.g., serum albumin) by conventional means such as autoclaving.

In order to obtain sterile-filterable particles of digitalis glycosides (i.e., particles<200 nm), the pharmacologically active agent is initially dissolved in a substantially water immiscible organic solvent (e.g., a solvent having less than about 5% solubility in water, such as, for example, chloroform) at high concentration, thereby forming an oil phase containing the pharmacologically active agent. Suitable solvents are set forth above. Unlike conventional methods for nanoparticle formation, a polymer (e.g. polylactic acid) is not dissolved in the solvent. The oil phase employed in the process of the present invention contains only the pharmacologically active agent dissolved in solvent.

Next, a water miscible organic solvent (e.g., a solvent having greater than about 10% solubility in water, such as, for example, ethanol) is added to the oil phase at a final concentration in the range of about 1%-99% v/v, more preferably in the range of about 5%-25% v/v of the total organic phase. The water miscible organic solvent can be selected from such solvents as ethyl acetate, ethanol, tetrahydrofuran, dioxane, acetonitrile, acetone, dimethyl sulfoxide, dimethyl formamide, methyl pyrrolidinone, and the like. alternatively, the mixture of water immiscible solvent with the water miscible solvent is prepared first, followed by dissolution of the pharmaceutically active agent in the mixture.

Next, in order to make the nanoparticles of digitalis glycosides, human serum albumin or any other suitable stabilizing agent as described above is dissolved in aqueous media. This component acts as a stabilizing agent for the formation of stable nanodroplets. Optionally, a sufficient amount of the first organic solvent (e.g. chloroform) is dissolved in the aqueous phase to bring it close to the saturation concentration. A separate, measured amount of the organic phase (which now contains the digitalis glycosides, the first organic solvent and the second organic solvent) is added to the saturated aqueous phase, so that the phase fraction of the organic phase is between about 0.5%-015% v/v, and more preferably between 1% and 8% v/v. Next, a mixture composed of micro and nanodroplets is formed by homogenization at low shear forces. This can be accomplished in a variety of ways, as can readily be identified by those of skill in the art, employing, for example, a conventional laboratory homogenizer operated in the range of about 2,000 up to about 15,000 rpm. This is followed by homogenization under high pressure (i.e., in the range of about 3,000 up to 30,000 psi). The resulting mixture comprises an aqueous protein solution (e.g., human serum albumin), the water insoluble digitalis glycosides, the first solvent and the second solvent. Finally, solvent is rapidly evaporated under vacuum to yield a colloidal dispersion system (digitalis glycosides and protein) in the form of extremely small nanoparticles (i.e., particles in the range of about 10 nm-200 nm diameter), and thus can be sterile-filtered. The preferred size range of the particles is between about 50 nm-170 nm, depending on the formulation and operational parameters.

Colloidal systems prepared in accordance with the present invention may be further converted into powder form by removal of the water therefrom, e.g., by lyophilization at a suitable temperature-time profile. The protein (e.g., human serum albumin) itself acts as a cryoprotectant, and the powder is easily reconstituted by addition of water, saline or buffer, without the need to use such conventional cryoprotectants as mannitol, sucrose, glycine, and the like. While not required, it is of course understood that conventional cryoprotectants may be added to invention formulations if so desired.

The polymeric shell containing solid or liquid cores of digitalis glycosides allows for the delivery of high doses of the pharmacologically active agent in relatively small volumes. In addition, the walls of the polymeric shell or coating are generally completely degradable in vivo by proteolytic enzymes (e.g., when the polymer is a protein), resulting in no side effects from the delivery system as is the case with current formulations.

According to this embodiment of the present invention, particles of substantially water insoluble digitalis glycosides have a cross-sectional diameter of no greater than about 10 microns. A cross-sectional diameter of less than 5 microns is more preferred, while a cross-sectional diameter of less than 1 micron is presently the most preferred for the intravenous route of administration.

Proteins contemplated for use as stabilizing agents in accordance with the present invention include albumins (which contain 35 cysteine residues), immunoglobulins, caseins, insulins (which contain 6 cysteines), hemoglobins (which contain 6 cysteine residues per α2 β2 unit), lysozymes (which contain 8 cysteine residues), immunoglobulins, α-2-macroglobulin, fibronectins, vitronectins, fibrinogens, lipases, and the like. Proteins, peptides, enzymes, antibodies and combinations thereof, are general classes of stabilizers contemplated for use in the present invention.

A presently preferred protein for use in the formation of a polymeric shell is albumin. Optionally, proteins such as α-2-macroglobulin, a known opsonin, could be used to enhance uptake of the shell encased particles of substantially water insoluble pharmacologically active agents by macrophage-like cells, or to enhance the uptake of the shell encased particles into the liver and spleen.

Specific antibodies may also be utilized to target the nanoparticles to specific locations.

In the preparation of invention compositions, a wide variety of organic media can be employed to suspend or dissolve the substantially water insoluble digitalis glycosides. Organic media contemplated for use in the practice of the present invention include any nonaqueous liquid that is capable of suspending or dissolving the pharmacologically active agent, but does not chemically react with either the polymer employed to produce the shell, or the pharmacologically active agent itself. Examples include vegetable oils (e.g., soybean oil, olive oil, and the like), coconut oil, safflower oil, cotton seed oil, sesame oil, orange oil, limonene oil, aliphatic, cycloaliphatic, or aromatic hydrocarbons having 4-30 carbon atoms (e.g., n-dodecane, n-decane, n-hexane, cyclohexane, toluene, benzene, and the like), aliphatic or aromatic alcohols having 2-30 carbon atoms (e.g., octanol, and the like), aliphatic or aromatic esters having 2-30 carbon atoms (e.g., ethyl caprylate (octanoate), and the like), alkyl, aryl, or cyclic ethers having 2-30 carbon atoms (e.g., diethyl ether, tetrahydrofuran, and the like), alkyl or aryl halides having 1-30 carbon atoms (and optionally more than one halogen substituent, e.g., CH₃Cl, CH₂Cl₂, CH₂Cl—CH₂Cl, and the like), ketones having 3-30 carbon atoms (e.g., acetone, methyl ethyl ketone, and the like), polyalkylene glycols (e.g., polyethylene glycol, and the like), or combinations of any two or more thereof.

Especially preferred combinations of organic media contemplated for use in the practice of the present invention typically have a boiling point of no greater than about 200° C., and include volatile liquids such as dichloromethane, chloroform, ethyl acetate, benzene, and the like (i.e., solvents that have a high degree of solubility for the pharmacologically active agent, and are soluble in the other organic medium employed), along with a higher molecular weight (less volatile) organic medium. When added to the other organic medium, these volatile additives help to drive the solubility of the pharmacologically active agent into the organic medium. This is desirable since this step is usually time consuming. Following dissolution, the volatile component may be removed by evaporation (optionally under vacuum).

Particles of pharmacologically active agent associated with a polymeric shell, prepared as described above, are delivered as a suspension in a biocompatible aqueous liquid. This liquid may be selected from water, saline, a solution containing appropriate buffers, a solution containing nutritional agents such as amino acids, sugars, proteins, carbohydrates, vitamins or fat, and the like.

In the present invention, efficacy of PSL nanoparticle formulations of the present invention with varying lipid compositions, particle size, and drug to lipid-protein ratio have been investigated on various systems such as human cell lines and animal models for cell proliferative activities. Furthermore, effects of PSL nanoparticle formulations and various drugs in their free form on the body weight of mice with different sarcomas and healthy mice without tumor have been investigated. Effects of PSL nanoparticle formulations and various drugs in their free form on the DNA fragmentation in different normal and tumor cells are investigated. These examples are not intended, however, to limit or restrict the scope of the present invention in any way and should not be construed as providing conditions, parameters, reagents, or starting materials which must be utilized exclusively in order to practice the art of the present invention.

It is known that certain anionic polysaccharides( Baba, 1988), such as dextran sulphate, pustulan sulphate stimulate cell-mediated T-cell dependent immune responses without stimulating anti-body mediated immune responses that are B-cell dependent. On the other hand, unmodified polysaccharides stimulate only B-cells and certain other polysaccharides are known to stimulate both T-cell and B-cell responses under certain conditions. The polysaccharides present in water extract of the plant Nerium Oleander has been shown to contain galacturonic acids similar to pectin. These polysaccharides are claimed to be immune stimulants. Thus the formulations of the present inventions can contain suitable polysaccharides such as pectin to provide the stimulant effect.

Compositions employing the novel compounds will contain a biologically effective amount of the compounds. As used herein a biologically effective amount of a compound or composition refers to an amount effective to alter, modulate or reduce tumor growth or related conditions. For intravenous administration, a satisfactory result may be obtained employing the compounds in an amount within the range of from about 0.001 mg/kg to about 5 mg/kg, preferably from about 0.002 mg/kg to about 2 mg/kg and more preferably from about 0.004 mg/kg to about 0.5 mg/kg alone or in combination with one or more additional anti-tumor compounds in an amount within the range from about 0.01 mg/kg to about 50 mg/kg, preferably from about 0.05 mg/kg to about 20 mg/kg and more preferably from about 0.1 mg/kg to about 10 mg/kg both being employed together in the same intravenous dosage form or in separate oral or intramuscular or intravenous dosage forms taken at the same time. The percentage of the compositions and preparations may, of course, be varied and may conveniently be between about 0.1 to about 50% of the weight of the unit. The amount of active compounds in such therapeutically useful compositions is such that a suitable dosage will be obtained.

The pharmaceutical formulations of oleandrin according to the present invention offer several advantages over the existing formulation of Nerium Oleander Extract administered parenterally. They can be intravenously administered and relatively high concentrations of oleandrin can be loaded into patients. Thus the frequency of dosage can be reduced. Thus within the spirit, the invention is related to improved formulations and methods of using the same when administering such formulations to patients. As mentioned herein above a number of excipients may be appropriate for use in the formulation which comprise the composition according to the present invention. The inclusion of excipients and the optimization of their concentration for their characteristics such as for example ease of handling or carrier agents will be understood by those ordinarily skilled in the art not to depart from the spirit of the invention as described herein and claimed herein below.

The invention will now be further described with reference to the following examples. These examples are intended to be merely illustrative of the invention and are not intended to be limiting.

### EXAMPLE 1

### Preparation of Liposome-Digitalis Glycoside Formulation

A lipid mixture containing HSPC:cholesterol:PEG2000-DSPE in a molar ratio of 55:40:5 was dissolved in a chloroform: ethanol (8.5.:1.5 vol/vol) mixture. For example, 3.55 g of HSPC, 1.39 g cholesterol and 1.33 g PEG2000-DSPE were dissolved in 30 mL of chloroform: ethanol (8.5:1.5 vol/vol) mixture. A chloroform-ethanol solution of oleandrin, in the range of 50-150 mg/mL was added to the above solution, resulting in a drug to lipid ratio of 1:10 (wt/wt). The above organic solution was added to the aqueous phase, with a pH of 8.0- 8.5, while mixing from 3000 to 10000 rpm. The mixture was subjected to either high-pressure microfluidization or homogenization. The pressure was varied between 20,000 and 30,000 psi. This resulted in a homogeneous and extremely fine oil-in-water emulsion. The emulsion was rapidly evaporated in an evaporator to a nanoparticle suspension. The evaporator pressure and the bath temperature during evaporation were 10-50 mm Hg and 30-70° C., respectively.

The particle size of the suspension was determined by photon correlation spectroscopy with the Malvern Zetasizer. The suspension was sterile-filtered through a 0.22 μm filter. The particle size of the suspension was between 30 and 220 nm. The suspension was frozen below −40° C. and lyophilized. The lyophilized cake was reconstituted prior to further use. The particle size did not change appreciably following lyophilization and reconstitution. In a similar manner, the liposomal formulations of neriifolin, odoroside A, odoroside H and proscillaridin A were prepared.

### EXAMPLE 2

### Preparation of PSL-Digitalis Glycoside Formulation

A lipid mixture containing HSPC:cholesterol:PEG2000-DSPE in a molar ratio of 55:40:5 was dissolved in a chloroform: ethanol (8.5.:1.5 vol/vol) mixture. For example, 3.55 g of HSPC, 1.39 g cholesterol and 1.33 g PEG2000-DSPE were dissolved in 30 mL of chloroform: ethanol (8.5:1.5 vol/vol) mixture. A chloroform-ethanol solution of oleandrin, in the range of 100-200 mg/mL was added to the above solution, resulting in a drug to lipid-protein ratio of 1:10 (wt/wt). A 1-10% human albumin solution was prepared. The pH of the solution was adjusted to 7.4. The above organic solution was added to the albumin phase while mixing from 3000 to 10000 rpm. The mixture was subjected to either high-pressure microfluidization or homogenization. The pressure was varied between 20,000 and 30,000 psi. This resulted in a homogeneous and extremely fine oil-in- water emulsion. The emulsion was rapidly evaporated in an evaporator to a nanoparticle suspension. The evaporator pressure and the bath temperature during evaporation were 10-50 mm Hg and 30-70° C., respectively.

The particle size of the suspension was determined by photon correlation spectroscopy with the Malvern Zetasizer. The suspension was sterile-filtered through a 0.22 μm filter. The particle size of the suspension was between 30 and 220 nm. The suspension was frozen below −40° C. and lyophilized. The lyophilized cake was reconstituted prior to further use. The particle size did not change appreciably following lyophilization and reconstitution.

In a similar manner, the PSL formulations ofneriifolin, odoroside A, odoroside H and proscillaridin A were prepared.

### EXAMPLE 3

### Preparation of PSL-Oleandrin Formulation

Lipid mixtures (Egg sphingomyelin:Phosphatidylcholine:cholesterol:PEG2000-DSPE=1:1:1:0.02 molar ratio) were dissolved in a chloroform:ethanol (9.5:0.05 vol/vol) mixture. A chloroform-ethanol solution of oleandrin, in the range of 100-200 mg/mL was added to the above solution, resulting in a drug to lipid ratio of 1:10 (wt/wt).

The above procedure described in Example 2 was employed to prepare PSL-Oleandrin. The particle size of the suspension before lyophilization and after reconstitution was between 50 and 220 nm.

### EXAMPLE 4

### Preparation of PSL-Oleandrin Formulation

The above procedure described in Example 2 was employed to prepare PSL-oleandrin formulation. However, instead of the lipid, PEG2000-DSPE, PEG2000-ceramide was used. The particle size of the suspension before lyophilization and after reconstitution was between 50 and 220 nm.

### EXAMPLE 5

### Preparation of PSL-Oleandrin Formulation

The above procedure described in Example 2 was employed to prepare PSL-Oleandrin. Lipid mixtures (Distearylphosphatidylcholine:cholesterol:PEG2000-ceramide=1.5:1:0.02 molar ratio) were dissolved in a chloroform:ethanol (8:2 vol/vol) mixture. The particle size of the suspension before lyophilization and after reconstitution was between 50 and 220 nm.

### EXAMPLE 6

### Preparation of PSL-Oleandrin Formulation

The above procedure described in Example 2 was employed to prepare PSL-Oleandrin. Lipid mixtures (egg phosphatidylcholine:cholesterol=55:45 molar ratio) were dissolved in a chloroform:ethanol (8:2 vol/vol) mixture or in chloroform or dicloromethane. The particle size of the suspension before lyophilization and after reconstitution was between 50 and 220 nm.

### EXAMPLE 7

### Preparation of PSL-Oleandrin Formulation

The above procedure described in Example 2 was employed to prepare PSL-Oleandrin formulation. Lipid mixtures (1,2-di(2,4-Tetradecadienoyl)-3-phosphatidylcholine:cholesterol=2:1 molar ratio) were dissolved in a chloroform:ethanol (8:2 vol/vol) mixture or in chloroform or dicloromethane. The particle size of the suspension before lyophilization and after reconstitution was between 50 and 220 nm.

### EXAMPLE 8

### Preparation of Nanoparticles of Oleandrin by High Pressure Homogenization

100 mg Oleandrin is dissolved in 10 ml methylene chloride. The solution was added to 81 ml of human serum abumin solution (1% w/v). The mixture was homogenized for 5 minutes at low RPM (Vitris homogenizer) in order to form a crude emulsion, and then transferred into a high pressure homogenizer (Avestin). The emulsification was performed at 9000-18,000 psi while recycling the emulsion for at least 5 cycles. The resulting system was transferred into a Rotary evaporator, and methylene chloride was rapidly removed at 40° C., at reduced pressure (30 mm Hg), for 20-30 minutes. The resulting dispersion was translucent, and the typical diameter of the resulting Oleandrin particles was 160-220 (Z-average, Malvern Zetasizer).

The dispersion was further lyophilized for 48 hrs. without adding any cryoprotectant. The resulting cake could be easily reconstituted to the original dispersion by addition of sterile water or saline. The particle size after reconstitution was the same as before lyophilization.

### EXAMPLE 9

### Preparation of Nanoparticles by Sonication

20 mg of Oleandrin is dissolved in 1.0 ml methylene chloride. The solution is added to 4.0 ml of human serum abumin solution (5% w/v). The mixture is homogenized for 5 minutes at low RPM (Vitris homogenizer, model:Tempest I.Q.) in order to form a crude emulsion, and then transferred into a 40 kHz sonicator cell. The sonicator is performed at 60-90% power at 0 degree for 1 min (550 Sonic Dismembrator). The mixture is transferred into a Rotary evaporator, and methylene chloride is rapidly removed at 40° C., at reduced pressure (30 mm Hg), for 20-30 minutes. The typical diameter of the resulting Oleandrin particles was 350-420 nm (Z-average, Malvern Zetasizer). The dispersion was further lyophilized for 48 hrs. without adding any cryoprotectant. The resulting cake could be easily reconstituted to the original dispersion by addition of sterile water or saline. The particle size after reconstitution was the same as before lyophilization.

### EXAMPLE 10

### Preparation of Less than 200 nm Sterile-Filterable Nanoparticles

10 mg of Oleandrin is dissolved in 0.55 ml chloroform and 0.05 ml ethanol. The solution is added to 29.4 ml of human serum abumin solution (1% w/v), which is presaturated with 1% chloroform. The mixture is homogenized for 5 minutes at low RPM in order to form a crude emulsion, and then transferred into a high pressure homogenizer (Avestin). The emulsification is performed at 9000-18,000 psi while recycling the emulsion for at least 6 cycles. The resulting system is transferred into a Rotary evaporator, and the chloroform is rapidly removed at 40° C., at reduced pressure (30 mm Hg), for 15-30 minutes.

The resulting dispersion is translucent, and the typical diameter of the resulting Oleandrin particles is 140-160 nm (Z-average, Malvern Zeta Sizer). The dispersion is filtered through a 0.22 micron filter (Millipore), without any significant change in turbidity, or particle size. HPLC analysis of the Oleandrin content revealed that more than 97% of the Oleandrin was recovered after filtration, thus providing a sterile Oleandrin dispersion. The sterile dispersion was further lyophilized for 48 hrs. without adding any cryoprotectant. The resulting cake could be easily reconstituted to the original dispersion by addition of sterile water or saline. The particle size after reconstitution was the same as before lyophilization.

### EXAMPLE 11

### Preparation of Less than 200 nm Sterile-Filterable Nanoparticles

225 mg Oleandrin is dissolved in 2.7 ml chloroform and 0.3 ml ethanol. The solution is added to 97 ml of human serum abumin solution (3% w/v). The mixture is homogenized for 5 minutes at low RPM (Vitris homogenizer) in order to form a crude emulsion, and then transferred into a high pressure homogenizer (Avestin). The emulsification is performed at 9000-18,000 psi while recycling the emulsion for at least 6 cycles. The resulting system is transferred into a Rotary evaporator, and the chloroform is rapidly removed at 40° C., at reduced pressure (30 mm Hg), for 15-30 minutes. The resulting dispersion is translucent, and the typical diameter of the resulting Oleandrin particles is 140-160 nm (Z-average, Malvern Zeta Sizer). The dispersion is filtered through a 0.22 micron filter (Sartorius, sartobran 300), without any significant change in turbidity, or particle size. HPLC analysis of the Oleandrin content typically revealed that 70-100% of the Oleandrin could be recovered after filtration, depending on the conditions employed. Thus, a sterile Oleandrin dispersion was obtained. The sterile dispersion was aseptically filled into sterile glass vials and lyophilized without adding any cryoprotectant. The resulting cake could be easily reconstituted to the original dispersion by addition of sterile water or saline. The particle size after reconstitution was the same as before lyophilization.

### References

Abe F and Yamauchi T: Phytochemistry, 31, 2459 (1992).

Allen T M, Hansen C, Martin F J, Redemann C, and Yau-Young A: Liposome containing a synthetic lipid derivative of poly(ethylene glycol) show prolonged circulation half-lives in vivo. Biochim. Biophys. Acta, 1066, 29-36 (1991).

Baba et al: Proc. Nat. Acad. Sci., USA, 85, 6132 (1988).

Baba et al: Antiviral Res., 9, 335 (1988).

Blum L M and Rieders F: Oleandrin distribution in a fatality from rectal and oral Nerium oleander extract administration. J Anal Toxicol, (1987) 11: 219-221.

Chopra R N, Nayar S L, and Chopra I C: Glossary of Indian Medicinal Plants, CSIR, New Delhi (1956), page 175.

Dey K L, and Bahadur R: Indigenous Drugs of India, International Book Publishers, India (1984).

Dobereiner H G, Kas J, Noppl D, Sprenger I, Sackmann E: Biophys. J. 65, 1396-140 (1993).

Drummond D C et al: Optimizing Liposomes for Delivery of Chemotherapeutic Agents to Solid Tumors. Pharmacological Reviews 51, 691-743, 1999 and the references cited therein.

Dymock W: Pharmacographia Indica, 2, 398, (1890).

Font-Quer P: (1979) Plantas medicinales. Barcelona, Ed. Labor S A.

Gabizon A. and Papahadjopoulos D: Liposome formulations with prolonged circulation time in blood and enhanced uptake by tumors. Proc. Natl. Acad. Sci. (Washington) 85, 6949-53 (1988).

Gordon K B, Tajuddin A, Guitart J, Kuzel, T M, Eramo L R, and Von Roenn J: Cancer, 75, 2169-73 (1995).

Hanada R, Abe F and Yamauchi T: Phytochemistry, 31, 2459 (1992).

Hanada R, Abe F and Yamauchi T: Phytochemistry, 31, 3183 (1992).

Hardin J W and Arena J M: (1974) Human poisoning from native a cultivated plants, 2nd ed. Kingsport, Tenn., Duke University Press.

Huxley A: ed-in-chief (1992) The New Horticultural Society dictionary of gardening, vol. 3. London, MacMillan.

Julliano R L and Stamp D: Pharmacokinetics of liposome-encapsulated anti-tumor drugs. Biochem. Pharm. 27, 21-27 (1978).

Kingsbury J M: (1964) Poisonous plants of the United States and Canada. Englewood Cliffs, N.J. Prentice Hall.

Kirtikar K R and Basu B D: Indian Medicinal Plants, International Book Ditributors, India (1987).

Krasso et al: Helv. Chim. Acta, 46, 1691 (1963).

Lasic D D, & Martin F: eds. (1995) Stealth Liposomes, CRC, BocaRaton, Fla.

Lasic D D and Paphadjopoulos D: Medical Applications of Liposomes, Amsterdam, Elsevier, 1998.

Lasic D D: Liposomes: From Physics to Applications. Amsterdam, Elsvier, 1993.

Lee et al: “Serum Albumin Beads: An Injectable, Biodegradable System for the Sustained Release of Drugs” Science, 213:233-235 (1981).

Leucuta et al: “Albumin microspheres as a drug delivery system for epirubicin: pharmaceutical, pharmacokinetic and biological aspects” International Journal of Pharmaceutics, 41:213-217 (1988).

Madden T D, Harrigan P R, Tai L C L, Bally M B, Mayer L D, Redelmeier T E, Loughrey H C, Tilcock C P S, Reinish L W and Cullis P R: The accumulation of drugs within large unilamellar vesicles exhibiting a proton gradient: a survey, Chem. Phys. Lipids, 53, 37-46 (1990).

Manna S K, Sah N K, Newman R A, Cisneros A, Aggarwal B B: Cancer Res, 60(14): 3838-47, 2000

McConkey D J, Lin Y, Nutt L K, Ozel H Z, Newman R A: Cancer Res, 60(14): 3807-12, 2000

Olson F, Hunt C, Szoka F C: Preparation of liposomes of defined size distribution by extrusion through polycarbonate membrane. BioChim. Biophys. Acta, 557, 9-23 (1979).

Olson F, Mayhew E, Maslow, Rustum Y and Szoka F: Characterization, toxicity and therapeutic efficacy of adriamycin entrapped in liposomes. Europ. J. Cancer. Clin. Oncol., 18, 167-76 (1982).

Pathak S, Multani A S, Narayan S, Kumar V, Newman R A: Anticancer Drugs, 11(6): 455-63, 2000

Pearn J: (1987) Oleander poisoning. In: Covacevich J, Davie P and Peam J: eds (1987) Toxic plants & animals; a guide for Australia. Brisbane, Queensland Museum, pp 37-49.

Rahman A., Kessler A., More N, Skies B, Rowden G, Wooley P and Schein P S: Liposomal protection of adriamycin induced cardiotoxicity in mice. Cancer Res., 40, 1532-37 (1982).

Reynolds J E F: ed (1989) Martindale: the extra pharmacopoeia, 29th ed. London, The Pharmaceutical Press.

Schvartsman S: (1979) Plantas venenosas. Sarvier Sao Paulo.

Sharma A, Sharma U S, and Straubinger R M: Paclitaxel-liposomes for intracavitary therapy of intraperitoneal P388 leukemia., Cancer Lett 107: 265-272 (1996).

Sharma A, Mayhew E, Bolcsak L, Cavanaugh C and Harmon P: Activity of paclitaxel liposome formulations against human ovarian tumor xenografts. Int J Cancer 71: 103-107 (1997).

Shaw D and Pearn J: (1979) Oleander poisoning. Med J Aust, 2: 267-269.

Siddiqui B S, Begum S, Hafeez F and Siddiqui S: Phytochemistry,28,1187 (1995).

Siddiqui B S, Begum S, Siddiqui S and Lichter W: Phytochemistry, 39,171 (1995).

Siddiqui S, Begum S, Hafeez F and Siddiqui B S: Phytochemistry, 28,1187 (1989).

Siddiqui S, Begum S, Siddiqui B S and Hafeez F: J. Nat. Products, 52, 229 (1988).

Siddiqui S, Hafeez F, Begum S and Siddiqui B S: J. Nat. Products, 49,1086 (1987).

Siddiqui S, Hafeez F, Begum S and Siddiqui B S: J. Nat. Products, 51, 229 (1988).

Siddiqui S, Hafeez F, Begum S, and Siddiqui B S: Phytochemistry, 26,237 (1987).

Siddiqui S, Siddiqui B S, Begum S, and Hafeez F: Plant Med., 55, 292 (1989).

Siddiqui S, Siddiqui B S, Hafeez F and Begum S: Planta Med., 53, 424 (1987).

Siddiqui S, Siddiqui B S, Hafeez F. and Begum S: Planta Med., 54, 232 (1988).

Smith J A, Madden T, Vijjeswarapu M, Newman R A: Biochem Pharmacol, 62(4): 469-72, 2001

Smith J A, Madden T, Vijjeswarapu M, Newman R A: Biochem Pharmacol, 62(4): 469-72, 2001

Suffness, M: (Ed.) Taxol, Science and Applications, Chapter 9, CRC (1995).

Taylor A., McKenna G F and Burlage H M: Texas Reports on Biology and Medicine, 14, 538 (1956).

Wang X, Plomley J B, Newman R A, Cisneros A: Anal Chem, 72(15): 3547-52, 2000

William S S, Alosco T R, Mayhew E, Lasic D D, Martin F J and Bankert R B: Arrest of human lung tumor xenograft growth in severe combined immunodeficient mice using doxorubicin encapsulated in sterically stabilized liposomes. Cancer Res., 53, 3964-67 (1993).

Woodle M C, Allen T M, Mayhew E, and Uster P S: In vivo efficacy of vincristine entrapped in long-circulating liposomes, Proc. Am. Assoc. Cancer Res., 33, 446 (1992).

Yeh J Y, Huang W J, Kan S F, Wang P S: Inhibitory effects of digitalis on the proliferation of androgen dependent and independent prostate cancer cells, J. Urol., 2001 166: 1937-42.

Stewart D, Mobasheri A and Djamgoz, M B A: Immuno-characterisation of Na⁺, K⁺-ATPase α-subunit(s) in rat prostate cancer cell lines, The Journal of Physiology (2000), 525P, pp. 96P.

Grimes J A, Fraser S P, Stephens G J, Downing J E, Laniado M E, Foster C S, Abel P D, Djamgoz M B: Differential expression of voltage-activated Na⁺ currents in two prostatic tumour cell lines: contribution to invasiveness in vitro., FEBS Lett 1995; 369:290-4.

Repke K R H, Schönfeld W, Weiland J, Megges R, Hache, A: In Design of Enzyme Inhibitors as Drugs; Sandler, M.; Smith, H. J., Eds.; Oxford University Press: Oxford, 1989; pp 435-502.

Thomas R E, Gray P, Andrews J: In Advances in Drug Research; Testa, L. B., Ed.; Academic Press: London, 1990; Vol. 19, pp 313-575.

Blaustein M P: Rev. Physiol. Biochem. Pharmacol. 1974, 70, 33-82.

Skou J C: Biochim. Biophys. Acta 1957, 23, 394-401.

Albers R W: Annu. Rev. Biochem. 1967, 36, 727-756.

Post R L, Kume S, Tobin T, Orcutt T, Sen A K: J. Gen. Physiol. 1969, 54, 306S-326S.

Geering K: Curr. Op. Nephrol. Hypertens. 1997, 6, 434-439.

Thomas R E: In Molecular Structure and Biological Activity of Steroids; Bohl M, Daux W L, Eds.; CRC Press: Boca Raton, 1992; pp 399-464.

Lee C O: Am. J. Physiol. 1985, 249, 367-378.

Gillis R A, Quest J A: In Cardiac Glycosides; Erdmann E, Greeff K, Skou J C, Eds.; Steinkopff: Darmstadt, 1986; pp 347-356.

Concepción P. Melero, Manuel M and Arturo S F: A Short Review on Cardiotonic Steroids and Their Aminoguanidine Analogues. Molecules 2000, 5, 51-81

Wald H, Scherzer P, Popovtzer MM: Enhanced renal tubular ouabain-sensitive ATPase in streptozotocin diabetes mellitus, Am J Physiol 1986; 251:F164-70

Jennings P E, Wilkins M R, West M J, Kendall M J, Barnett A H: Sodium transport across erythrocyte membranes in diabetes mellitus. Sodium transport across erythrocyte membranes in diabetes mellitus, Diabetes Res 1986; 3:407-10

Kjeldsen K, Braendgaard H, Sidenius P, Larsen J S, Norgaard A: Diabetes decreases Na+-K+ pump concentration in skeletal muscles, heart ventricular muscle, and peripheral nerves of rat, Diabetes 1987; 36:842-8

Baldini P, Incerpi S, Lambert-Gardini S, Spinedi A, Luly P: Membrane lipid alterations and Na+-pumping activity in erythrocytes from IDDM and NIDDM subjects, Diabetes 1989; 38:825-31

Kuriyama S, Tokudome G, Tomonari H, Utsunomiya Y, Matsui K, Hashimoto T, Sakai O: Differential regulation of cation transport of vascular smooth muscle cells in a high glucose concentration milieu, Diabetes Res Clin Pract 1994; 24:77-84

Raccah D, Dadoun F, Coste T, Vague P: Decreased Na/K ATPase ouabain binding sites in red blood cells of patients with insulin-dependent diabetes and healthy north African control subjects: relationship with diabetic neuropathy, Horm Metab Res 1996; 28:128-32

Zolese G, Rabini R A, Fumelli P, Staffolani R, Curatola A, Kvasnicka P, Kotyk A, Cester N, Mazzanti L: Modifications induced by insulin-dependent diabetes mellitus on human placental Na+/K+-adenosine triphosphatase, J Lab Clin Med. 1997; 130:352-3.

Gerbi A, Maixent J M, Barbey O, Jamme I, Pierlovisi M, Coste T, Pieroni G, Nouvelot A, Vague P, Raccah D: Alterations of Na, K-ATPase isoenzymes in the rat diabetic neuropathy: protective effect of dietary supplementation with n-3 fatty acids, J Neurochem 1998; 71:732-40

Wild G, Thompson J A, Searles L, Turner R, Hasan J, Thomson A B: Small intestinal Na+, K+-adenosine triphosphatase activity and gene expression in experimental diabetes mellitus, Dig Dis Sci 1999; 44:407-14

Takigawa T, Yasuda H, Terada M, Maeda K, Haneda M, Kashiwagi A, Kitasato H, Kikkawa R: Increases in K+ conductance and Ca2+ influx under high glucose with suppressed Na+/K+-pump activity in rat myelinated nerve fibers, Neuroreport 2000; 11:2547-51

Sennoune S, Gerbi A, Duran M J, Grillasca J P, Compe E, Pierre S, Planells R, Bourdeaux M, Vague P, Pieroni G, Maixent J M: Effect of streptozotocin-induced diabetes on rat liver Na+/K+-ATPase, Eur J Biochem 2000; 267:2071-8.

Shiratori O: Growth inhibitory effect of cardiac glycosides and agly cones on neoplastic cells: in vitro and in vivo studies. Gann 1967, 58:521-528

Repke K R H: The role of the Na+/K+ pump in normal and cancer cell proliferation. In: Biomemebranes, Basic and Medical Research (Edited by Benga G, Tager J M) Berlin, Springer Verlag 1988, 161-176

Repke K R, Schon R, Megges R, Wetland J, Nissen E, Matthes E: Potential suitability of Na+/K(+)-transporting ATPase in pre-screens for anti-cancer agents. Anticancer Drug Des 1995, 10:177-87

Cassady J M, Suffness M M: Terpenoid antitumor targets. In: Anti cancer agents based on natural product models (Edited by Cassady J M, Douros J D) New York, Academic Press 1980, 201-269

Haux J: Digitoxin is a potential anticancer agent for several types of cancer. Med Hypotheses 1999, 53:543-548

Haux J, Lam M, Marthinsen A B L, Strickert T, Lundgren S: Digitoxin, in non toxic concentrations, induces apoptotic cell death in Jurkat T cells in vitro. Z Onkol 1999, 31:14-20.

Haux J, Solheim O, Isaksen T, Angelsen A: Digitoxin, in non-toxic concentrations, inhibits proliferation and induces cell death in prostate cancer cell lines. Z Onkol 2000, 32:11-16

Kawazoe N, Aiuchi T, Masuda Y, Nakajo S, Nakaya K: Induction of apoptosis by bufalin in human tumor cells is associated with a change of intracellular concentration of Na+ ions. J Biochem (Tokyo) 1999, 126:278-286

Johansson S, Lindholm P, Gullbo J, Larsson R, Bohlin L, Claeson P: Cytotoxicity of digitoxin and related cardiac glycosides in human tumor cells. Anticancer Drugs 2001-, 12:475-483

What we claim are:

1. A method for the preparation of a variety of liposomal digitalis glycosides compositions for the use of treating cell-proliferative diseases in humans and mammals, the method comprising:
a) obtaining in a hydrophobic solvent a liposome forming mixture comprising a phosphalipid and cholesterol;
b) adding a hydrophobic solution of digitalis glycoside to the mixture to form a drug:lipid ratio of about 1:10;
c) hydrating the mixture with high speed mixing to form a suspension in aqueous solution;
d) subjecting the suspension to high pressure microfluidization;
e) evaporating the organic solvent under reduced pressure to form a lipid suspension in aqueous solution;
f) filtering the suspension to remove any precipitated digitalis glycoside.

2. A method for the preparation of a variety of liposomal digitalis glycosides compositions for the use of treating cell-proliferative diseases in humans and mammals, the method comprising:
a) obtaining a hydrophobic solvent comprising dissolved HSPC;
cholesterol and PEG 2000-DSPE at a 55:40:5 molar ratio;
b) adding a hydrophobic solution of digitalis glycoside to the mixture to form a drug:lipid ratio of about 1:10;
c) hydrating the mixture with high speed mixing to form a suspension in aqueous solution;
d) subjecting the suspension to high pressure microfluidization;
e) evaporating the organic solvent under reduced pressure to form a lipid suspension in aqueous solution;
f) filtering the suspension to remove any precipitated digitalis glycoside.

3. The liposomal digitalis glycoside compositions prepared by the method of claim 1 or 2 defined further as having reduced toxicity, high drug to lipid ratio, long-circulating time in the bloodstream and the ability to deliver the drug to the tumor sites.

4. The liposomal digitalis glycoside composition prepared by the method of claim 1 or 2, defined further as being effective in reducing the growth of cancers or the incidence of metastases.

5. The liposomal digitalis glycoside prepared by the method of claim 1 or 2 defined further as being effective in treating cell-proliferative and other diseases in a warm-blooded animals.

6. The liposomal digitalis glycoside composition prepared by the method of claim 1 defined further as being prepared using lipids including at least one hydrogenated soy phosphatidylcholine (HSPC), egg phosphatidylcholine (EPC, phosphatidylethanolamine (PE), phosphatidylglycerol(PG), phosphatidylinsitol (PI), monosialoganglioside sphingomyelin (SPM); derivatized vesicle forming lipids such as poly(ethylene glycol)-derivatized distearoylphosphatidylethanolamine (PEG-DSPE) distearoylphosphatidylcholine (DSPC), dimyristoylphospha-tidylcholine (DMPC), and dimyristoylphosphatidylglycerol (DMPG), poly(ethylene glycol)-derivatized ceramides (PEG-CER) and mixtures thereof.

7. The liposomal digitalis glycoside composition made by the method of claim 1 or 2 wherein the phospholipids are synthetic or derived from natural services.

8. The liposomal digitalis glycoside composition prepared by the method of claim 1 or 2, as defined further as having a drug to lipid weight ratio between 0.01 and 1, preferably between 0.1 and 1.

9. The liposomal composition prepared by the method of claim 1 or 2 wherein digitalis glycoside is derived from plants or animals or by synthetic routes.

10. The liposomal digitalis glycoside composition prepared by the method of claim 1 or 2 defined further as being prepared by solvent evaporation of digitalis glycosides, cholesterol, and lipids followed by hydration and high shear stress to reduce particle size of the liposomal-digitalis glycosides.

11. The method of claim 11 where the ratio by weight of digitalis glycosides to cyclodextrin is 0.01 to 10.

12. The method of claim 1 defined further as yielding liposomal digitalis glycoside having a size less than 220 nm, preferably 80-160 nm, most preferably between about 100-120 nm.

13. The liposomal digitalis glycoside composition prepared by the method of claim 1 or 2 defined further as being sterile filtered through a 0.22 μm filtered and lyophilized.

14. The liposomal digitalis glycoside composition prepared by the method of claim 13 defined further as being lyophilized in the form of a cake in vials using cryoprotectants such as sucrose, mannitol, trehalose or the like.

15. The composition prepared by the method of claim 14 wherein the lyophized cake is defined further as being reconstitutable to the original liposomes, without modifying liposome particle size.

16. The liposomal digitalis glycoside composition prepared by the method of claim 1 or 2 defined further as being deliverable in the form of nanoparticles or suspensions, by slow infusions or by bolus injection or by other parenteral or oral delivery routes.

17. A pharmaceutical formulation of digitalis glycosides active for in vivo delivery, said formulation is prepared by subjecting a mixture comprising:
a) an organic phase containing digitalis glycoside dispersed therein, and
b) aqueous medium containing biocompatible polymer,
wherein said mixture contains substantially no surfactants, to high shear conditions in a high pressure homogenizer at a pressure in the range of about 3,000 up to 30,000 psi.

18. The pharmaceutical formulation according to claim 17 further comprising removing said organic phase from said mixture.

19. The pharmaceutical formulation according to claim 17 further comprising removing said aqueous phase from said mixture.

20. The pharmaceutical formulation to claim 17 wherein said organic phase has a boiling point of no greater than about 200.degree. C.

21. The pharmaceutical formulation according to claim 20 wherein said organic phase is selected from soybean oil, coconut oil, olive oil, safflower oil, cotton seed oil, sesame oil, orange oil, limonene oil, aliphatic, cycloaliphatic or aromatic hydrocarbons having 4-30 carbon atoms, aliphatic or aromatic alcohols having 2-30 carbon atoms, aliphatic or aromatic esters having 2-30 carbon atoms, alkyl, aryl, or cyclic ethers having 2-30 carbon atoms, alkyl or aryl halides having 1-30 carbon atoms, optionally having more than one halogen substituent, ketones having 3-30 carbon atoms, polyalkylene glycol, or combinations of any two or more thereof.

22. The pharmaceutical formulation according to claim 20 wherein said organic phase comprises a mixture of a substantially water immiscible organic solvent and a water soluble organic solvent.

23. The pharmaceutical formulation according to claim 17 wherein said biocompatible polymer is a naturally occurring polymer, a synthetic polymer, or a combination thereof.

24. The pharmaceutical formulation according to claim 17 wherein said biocompatible polymer is capable of being crosslinked by disulfide bonds.

25. The pharmaceutical formulation according to claim 23 wherein said naturally occurring polymers are selected from proteins, peptides, polynucleic acids, polysaccharides, proteoglycans or lipoproteins.

26. The pharmaceutical formulation according to claim 23 wherein said synthetic polymers are selected from synthetic polyamino acids containing cysteine residues and/or disulfide groups; polyvinyl alcohol modified to contain free sulfhydryl groups and/or disulfide groups; polyhydroxyethyl methacrylate modified to contain free sulfhydryl groups and/or disulfide groups; polyacrylic acid modified to contain free sulfhydryl groups and/or disulfide groups; polyethyloxazoline modified to contain free sulfhydryl groups and/or disulfide groups; polyacrylamide modified to contain free sulfhydryl groups and/or disulfide groups; polyvinyl pyrrolidinone modified to contain free sulfhydryl groups and/or disulfide groups; polyalkylene glycols modified to contain free sulfhydryl groups and/or disulfide groups; polylactides, polyglycolides, polycaprolactones, or copolymers thereof, modified to contain free sulfhydryl groups and/or disulfide groups; as well as mixtures of any two or more thereof.

27. The pharmaceutical formulation according to claim 17 wherein said high shear conditions comprise contacting said organic phase and said aqueous medium in a high pressure homogenizer at a pressure in the range of about 6,000 up to 25,000 psi.

28. The pharmaceutical formulation according to claim 17 wherein said biocompatible polymer is the protein albumin.

29. The pharmaceutical formulation according to claim 17 wherein said aqueous medium is selected from water, buffered aqueous media, saline, buffered saline, solutions of amino acids, solutions of sugars, solutions of vitamins, solutions of carbohydrates, or combinations of any two or more thereof.

30. The pharmaceutical formulation according to claim 17 wherein said high shear conditions produce particles comprising said pharmacologically active agent coated with said biocompatible polymer.

31. The pharmaceutical formulation according to claim 30 wherein said particles have an average diameter of less than 1 micron.

32. The pharmaceutical formulation according to claim 30 wherein said particles have an average diameter of less than 200 nm.

33. The pharmaceutical formulation according to claim 32 wherein said mixture is sterile filtered.

34. The pharmaceutical formulation according to claim 30 wherein said particles are amorphous, crystalline, or a mixture thereof.

35. The pharmaceutical formulation according to claim 34 wherein said particles are substantially amorphous.

36. The pharmaceutical formulation for the preparation of digitalis glycosides for in vivo delivery in the form of sterile-filterable particles, said formulation is prepared by subjecting a mixture comprising:
(a) an organic phase containing digitalis glycoside dispersed therein, wherein said organic phase comprises a mixture of a substantially water immiscible organic solvent and a water soluble organic solvent, and
(b) aqueous medium containing biocompatible polymer,
wherein said mixture contains substantially no surfactants, to high shear conditions in a high pressure homogenizer at a pressure in the range of about 3,000 up to 30,000 psi.

37. The pharmaceutical formulation according to claim 36 further comprising removing said organic phase from said mixture.

38. The pharmaceutical formulation according to claim 36 further comprising filtering said mixture through a 0.22 micron filter.

39. The pharmaceutical formulation according to claim 36 further comprising removing said aqueous phase from said mixture.

40. The pharmaceutical formulation according to claim 36 wherein said high shear conditions produce amorphous particles, crystalline particles, or a mixture thereof.

41. The pharmaceutical formulation according to claim 40 wherein said particles are substantially amorphous.

42. A pharmaceutical composition comprising: a protein-stabilized liposome containing digitalis glycosides, the pharmaceutical composition is prepared by the method comprising:
a) preparing an organic solution of liposome-forming phospholipids, cholesterol and PEG-phospholipid;
b) adding at least one digitalis glycoside dissolved in an organic solvent;
c) mixing the phospholipid and pharmaceutical agent mixture at a ratio of about one part drug to ten parts lipid;
d) subjecting said mixture to infusion into a one to ten percent aqueous solution of a protein in an aqueous medium a pH between about 7.2 and 7.6;
e) after agitating said mixture at between about 3000 and about 10,000 revolutions per minute, said mixture is subjected to high pressure microfluidization or homogenization at 20,000 to 30,000 PSI, which results in a fine oil and water emulsion;
f) evaporating the organic solvent under reduced pressure to form a lipid suspension in aqueous solution;
g) filtering the suspension to remove any precipitated digitalis glycoside.

43. The method of claim 42 defined further as having a final step of lyophilization to produce a powder suitable for reconstituting a protein stabilized liposomal formulation in an aqueous suspension.

44. The protein-stabilized liposomal (PSL) formulations containing digitalis glycosides as nanoparticle suspensions or lyophilized powders for the use in treating cell-proliferative and other diseases in humans and mammals, said formulation prepared by the method of claims 42 or 43.

45. The protein-stabilized liposomal (PSL) formulation prepared according to claim 42 defined further as having reduced toxicity, high drug to lipid ratio, long-circulating time in the bloodstream and ability to deliver the drug to the target sites.

46. The protein-stabilized liposomal (PSL) formulations containing digitalis glycosides prepared according to claim 42 defined further as effective in reducing the growth of cancers or reducing the incidence of metastases.

47. The protein-stabilized liposomal (PSL) formulations containing digitalis glycosides prepared according to claim 42 defined further as effective in treating cell-proliferative and other diseases in a warm-blooded animals.

48. The protein-stabilized liposomal (PSL) formulations prepared according to claim 42 defined further as prepared using lipids such as hydrogenated soy phosphatidylcholine (HSPC), egg phosphatidylcholine (EPC, phosphatidylethanolamine (PE), phosphatidylglycerol(PG), phosphatidylinsitol (PI), monosialogangolioside and spingomyelin (SPM); the derivatized vesicle forming lipids such as poly(ethylene glycol)-derivatized distearoylphos-phatidylethanolamine (PEG-DSPE) and poly(ethylene glycol)-derivatized ceramides (PEG-CER). The phospholipid can be either synthetic or derived from natural sources such as egg or soy. The phospholipids can also be distearoylphosphatidylcholine (DSPC), dimyristoylphosphatidylcholine (DMPC), and dimyristoylphosphatidylglycerol (DMPG) and proteins such as human serum albumin, bovine serum albumin, and erythroprotein.

49. The protein-stabilized liposomal (PSL) formulations prepared according to claim 42, wherein the cardiac glycoside to lipid-protein weight ratio varies between 0.01 and 1, preferably between 0.05 and 1.

50. The Pharmaceutical formulations prepared according to claim 42 wherein the liposome-forming materials prepared from plants or by synthetic routes.

51. The PSL formulations prepared according to claim 42 defined further as prepared by solvent evaporation of digitalis glycoside, cholesterol, and lipids followed by hydration and high shear stress to reduce the particle size of the PSL nanoparticles.

52. The method of claim 42 defined further as yielding PSL nanoparticles having size less than 220 nm, preferably 80-160 nm, most preferably between about 100-120 nm.

53. The PSL formulations prepared according to claim 42 defined further as being sterile filtered through a 0.22 μm filtered and lyophilized.

54. The PSL formulations prepared according to claim 53 defined further as being sterile-filtered and lyophilized in the form of a cake in vials using cryoprotectants such as sucrose, mannitol, trehalose or the like.

55. The formulations of claim 54 wherein the lyophized cake can be reconstituted to the original PSL formulations, without modifying the particle size of PSL nanoparticles.

56. The PSL formulations prepared according to claim 42 defined further as being deliverable in the form of nanoparticles or suspensions by slow infusions or by bolus injection or by other parenteral or oral delivery routes.

57. The pharmaceutical formulation according to claim 42 wherein said protein is selected from albumins, immunoglobulins, caseins, insulins, hemoglobins, lysozymes, immunoglobulins, α-2-macroglobulin, fibronectins, vitronectins, fibrinogens, lipases, enzymes and antibodies.

58. The pharmaceutical formulation according to claim 42 wherein said protein is albumin.

59. The digitalis glycoside in claims 1, 2, 17, 36, 42 is selected from the group consisting of oleandrin, neriifolin, odoroside A and H, ouabain (G-strophantin), cymarin, sarmentocymarin, periplocymarin, K-strophantin, thevetin A, cerberin, peruvoside, thevetosin, thevetin B, tanghinin, deacetyltanghinin, echujin, hongheloside G, honghelin, periplocin, strophantidol, nigrescin. uzarin, calotropin, cheiroside A, cheirotoxin, euonoside, euobioside, euomonoside, lancetoxin A and B, kalanchoside, bryotoxin A-C, bryophyllin B, cotiledoside, tyledoside A-D, F and G, orbicuside A-C, alloglaucotoxin, corotoxin, coroglaucin, glaucorin, scillarene A and B, scilliroside, scilliacinoside, scilliglaucoside, scilliglaucosidin, scillirosidin, scillirubrosidin, scillirubroside, proscillaridin A, rubelin, convalloside, convallatoxin, bovoside A, glucobovoside A, bovoruboside, antiarin A, helleborin, hellebrin, adonidin, adonin, adonitoxin, thesiuside, digitoxin, gitoxin, gitalin, digoxin, F-gitonin, digitonin, lanatoside A-C, bufotalin, bufotalinin, bufotalidin, pseudobufotalin, acetyl-digitoxin, acetyl-oleandrin, beta-methyldigoxin and alpha-methyldigoxin.

60. The pharmaceutical formulation according to claim 42 wherein said organic phase is selected from soybean oil, coconut oil, olive oil, safflower oil, cotton seed oil, sesame oil, orange oil, limonene oil, aliphatic, cycloaliphatic or aromatic hydrocarbons having 4-30 carbon atoms, aliphatic or aromatic alcohols having 2-30 carbon atoms, aliphatic or aromatic esters having 2-30 carbon atoms, alkyl, aryl, or cyclic ethers having 2-30 carbon atoms, alkyl or aryl halides having 1-30 carbon atoms, optionally having more than one halogen substituent, ketones having 3-30 carbon atoms, polyalkylene glycol, or combinations of any two or more thereof.

61. The digitalis glycoside in claims 1, 2, 17, 36 and 42 is Oleandrin

62. The digitalis glycoside in claims 1, 2, 17, 36 and 42 is Neriifolin.

63. The digitalis glycoside in claims 1, 2, 17, 36 and 42 is Odoroside A.

64. The digitalis glycoside in claims 1, 2, 17, 36 and 42 is Odoroside H.

65. The digitalis glycoside in claims 1, 2, 17, 36 and 42 is Digoxin.

66. The digitalis glycoside in claims 1, 2, 17, 36 and 42 is Digitoxin.

67. The digitalis glycoside in claims 1, 2, 17, 36 and 42 is Proscillaridin A.
